(19) United States
(12) Patent Application Publication
(10) Pub. No.: US 2026/0077091 A1
(43) Pub. Date: Mar. 19, 2026

(54) INJECTABLE COLLAGEN IMPLANTS AND COMPOSITIONS AND RELATED SYSTEMS, AND METHODS OF PREPARATION AND USE THEREOF

(71) Applicant: Miach Orthopaedics, Inc., Westborough, MA (US)

(72) Inventors: Stephen WOHLERT, Flemington, NJ (US); Rita PAPARAZZO, Hopkinton, MA (US)

(73) Assignee: Miach Orthopaedics, Inc., Westborough, MA (US)

(21) Appl. No.: 19/397,684

(22) Filed: Nov. 21, 2025

Related U.S. Application Data
(63) Continuation of application No. 19/280,826, filed on Jul. 25, 2025.
(60) Provisional application No. 63/676,038, filed on Jul. 26, 2024.
(60) Provisional application No. 63/740,568, filed on Dec. 31, 2024.

(51) Int. Cl. A61L 26/00; A61F 2/08; A61F 2/46

(52) U.S. Cl. CPC A61L 26/0033; A61F 2/0805; A61F 2/0811; A61F 2/4618; A61F 2002/0823; A61F 2002/0882

(57) ABSTRACT

Described herein are methods for repairing tissues (e.g., a meniscus, anterior cruciate ligament, rotator cuff, etc.) using collagen implants suitable. For example, the collagen implants are injected into a repair site to heal the tissue. Further described are collagen slurries, injectable collagen implants and methods of preparation thereof.

### CROSS-REFERENCE TO RELATED APPLICATIONS

The present application is a continuation of U.S. patent application Ser. No. 19/280,826, filed Jul. 25, 2025, which claims the benefit of and priority to U.S. Prov. Patent Application No. 63/740,568, filed Dec. 31, 2024; and U.S. Provisional Patent Application. No. 63/676,038, filed on Jul. 26, 2024, the entire contents of which are incorporated by reference into the present application.

### FIELD

The present disclosure relates to injectable collagen implants and compositions and related systems and methods of preparation and use thereof.

### BACKGROUND

While the body has efficient processes for healing most damaged tissue, tissue such as intra-articular tissue often fails to heal after an injury. The tissue outside of joints heals by forming a fibrin clot, which connects the ruptured tissue ends and is subsequently remodeled to form scar, which heals the tissue. Inside a synovial joint, a fibrin clot either fails to form or is quickly lysed after injury to the knee, thus preventing joint arthrosis and stiffness after minor injury. Joints contain synovial fluid which, as part of normal joint activity, naturally prevent clot formation in joints. This fibrinolytic process results in premature loss of the fibrin clot scaffold and disruption of the healing process for tissues within the joint or within intra-articular tissues. Enhancing healing of ligaments using growth factors has been an area of great interest and research.

Recent developments in scaffolds for tissue repair show promise in terms of improved health outcomes, faster healing, and few long-term adverse effects. Developing scaffolds and the ability to inject repair material directly to the site of the tissue defect or tear can improve healing.

### SUMMARY

According to various embodiments described herein are collagen slurry implant compositions. The compositions may include collagen in an amount of about 40 w/w % to about 70 w/w %, a buffer in an amount of about 15 w/w % to about 30 w/w %, at least one salt in an amount of about 2 w/w % to about 5 w/w %, at least one electrolyte in an amount of about 5 w/w % to about 11 w/w %, and glycosaminoglycan in an amount of less than about 2 w/w %, or about 0.001 w/w % to less than about 2 w/w %, or any individual value or sub-range within these ranges. The slurries are suitable to self-assemble into a collagen implant, which may be divided into particles (e.g., pieces, granules, powder, etc.) and injected into a tissue repair site.

Further described herein according to embodiments, are collagen implant configured for injection into a target tissue. In some embodiments, the collagen implants comprise plurality of particles. Each particle may include collagen in an amount of about 40 w/w % to about 70 w/w %, at least one salt in an amount of about 2 w/w % to about 5 w/w %, at least one electrolyte in an amount of about 5 w/w % to about 11 w/w %, and glycosaminoglycan in an amount of less than about 2 w/w %, or about 0.001 w/w % to less than about 2 w/w %, or any individual value or sub-range within these ranges. In some embodiments, collagen implants comprise the collagen in an amount of about 40 w/w % to about 70 w/w %. In embodiments, the collagen is in an amount of greater than 400 mg/g, or about 400 mg/g to about 10,000 mg/g. In various embodiments, the at least one glycosaminoglycan is in an amount of less than about 2 w/w %, or about 0.001 w/w % to less than about 2 w/w %, or any individual value or sub-range within these ranges. In embodiments, the at least one glycosaminoglycan is in an amount of greater than 100 μg/g, or greater than about 100 μg/g to about 8,000 μg/g. In embodiments, the injectable collagen implants further comprise DNA in amount of less than 50,000 ng/g, or about 5 ng/g to less than about 50,000 ng/g. In at least one embodiment, the injectable collagen implants comprise a phospholipid and having a phospholipid count of less than 3,000 μM/g, or about 1 μM/g to less than about 3,000 μM/g. In various embodiments, the injectable collagen implants further comprise pepsin in an amount of less than 12.5 mg/g, or about 0.1 mg/g to less than about 12.5 mg/g.

In yet further embodiments described herein are methods for preparing a collagen slurry. Such methods may include performing a salt extraction of a source tissue to produce a salt extracted collagen; performing a detergent extraction of the salt extracted collagen to produce a detergent extracted collagen; performing an enzyme digestion of the detergent extracted collagen to produce an enzyme extracted collagen; and performing an acid solubilization of the enzyme extracted collagen to produce a purified collagen slurry.

According to various embodiments described are methods of making a collagen implant configured for injection into a tissue. These methods may include preparing a collagen slurry according to embodiments herein; heating the collagen slurry to form a self-assembled collagen slurry; and freeze-drying the self-assembled collagen slurry to produce the collagen implant. In some embodiments, the collagen implant is in the form of a plurality of particles. In some embodiments, the collagen implant is in the form of a semi-solid or liquid slurry material.

Further described herein according to embodiments, are tissue repair systems. Tissue repair systems may include a collagen implant configured for injection into a target tissue according embodiments herein; and a delivery device configured to deliver the collagen implant to the target tissue.

In further embodiments, are delivery devices for tissue repair, comprising: an elongated body having a proximal end, a distal end, a channel that extends from the proximal end to the distal end, and an actuator in the channel; and a collagen implant configured for injection into a target tissue according to embodiments herein. In some embodiments, the collagen implant is housed within the delivery device. The actuator may be configured to cause the collagen implant to exit the distal end of the body.

In yet further embodiments, are tissue repair kits, comprising: a tissue repair system, according to embodiments herein. The tissue repair kits may include a collagen implant configured for injection into a target tissue according to embodiments, and a delivery device configured to deliver the collagen implant to the target tissue together with instructions for use.

Methods for repairing a tissue are described. Such methods include forming a portal in a repair site of the tissue; inserting a delivery device containing a collagen implant according to embodiments herein through the portal such that a distal end of the delivery device is positioned close to the tissue; and manipulating the delivery device to release the collagen implant from the distal end of the delivery device and to the tissue such that the collagen implant rests within or near the tissue.

According to various embodiments are methods for repairing a meniscus, comprising: forming a portal in a knee joint to access a lateral or medial meniscus, wherein the lateral or medial meniscus has a rupture or tear; inserting a delivery device into the portal so that a distal end of the delivery device is positioned close to the lateral or medial meniscus; causing a collagen implant according to any one of claims 18 to 50 to exit the distal end of the delivery device in order to position it on or near the rupture or tear of the lateral or medial meniscus; and securing the collagen implant in place at the rupture or tear of the lateral or medial meniscus.

Embodiments further include methods for repairing an anterior cruciate ligament (ACL), comprising: drilling a tibial tunnel in a tibia: drilling a femoral tunnel in a femur so that the femoral tunnel and tibia tunnels are aligned; placing a bone fixation device on a femoral cortex, wherein a first and second set of sutures are attached to the bone fixation device and pass through the femoral tunnel; securing the first set of sutures onto the ACL with a rupture or tear; injecting a collagen implant according to embodiments herein at or near the second set of sutures; passing the first and second set of sutures, attached to a second bone fixation device, through the tibial tunnel; securing the bone fixation device in place on the tibial cortex; and observing growth of an ACL tissue and meniscus at or near a rupture or tear of the ACL and the meniscus.

In further embodiments are methods for repairing one or one tears or ruptures in an anterior cruciate ligament (ACL). Such methods may include coupling a first suture assembly and a second suture assembly to a bone fixation device; securing a first portion of the first set of sutures to a ruptured or torn anterior cruciate ligament; positioning the bone fixation device on a surface of the femur so that the first and second sets of sutures extend through a femoral tunnel; injecting a collagen implant according to embodiments herein proximate the ACL and a lateral or medial meniscus; securing with a second bone fixation device the second set of sutures to a surface of the tibia so that the second set of sutures extends through a tibial tunnel; and observing growth of an ACL tissue and meniscus at or near a rupture or tear of the ACL and the meniscus.

In one or more embodiments, further described are methods of repairing an ACL, comprising: securing a first portion of a first suture assembly to a ruptured or torn anterior cruciate ligament; securing a second portion of the first suture assembly relative to a femur such that the first set of sutures extends from the ruptured or torn anterior cruciate ligament and through a femoral tunnel to a bone fixation device; securing a first portion of a second set of sutures relative to a tibia so that a suture tail of the second set of sutures extends through a tibial tunnel into a region near the anterior cruciate ligament; securing a second portion of the second set of sutures relative to a femur at the bone fixation device so that the second set of sutures extends into a femoral tunnel alongside or adjacent the anterior cruciate ligament; injecting a collagen implant according to embodiments herein at a location adjacent the rupture or torn portion of the ACL; permitting blood from the collagen implant to emanate from the collagen implant into the region near the anterior cruciate ligament and a tear or rupture in either or both of a lateral meniscus and a medial meniscus; and observing healing of the rupture or tear of ACL and the lateral meniscus or medial meniscus.

In yet further embodiments are methods for repairing a rotator cuff tendon, comprising: attaching a first fixation device to a humerus; attaching a second fixation device to the rotator cuff tendon; connecting a flexible construct to the two fixation devices; injecting a collagen implant according to embodiments herein on or near the rotator cuff such that the collagen implant rests between torn ends of the rotator cuff tendon.

### BRIEF DESCRIPTION OF THE DRAWINGS

The foregoing summary, as well as the following detailed description of illustrative embodiments of the present application, will be better understood when read in conjunction with the appended drawings. For the purposes of illustrating the present application, there is shown in the drawings illustrative embodiments of the disclosure. It should be understood, however, that the application is not limited to the precise arrangements and instrumentalities shown:

FIG. 1A is a front view illustration of a shoulder joint;

FIG. 1B is a front view illustration of a shoulder joint with a partial rotator cuff tear;

FIG. 1C is a front view illustration of a shoulder joint with a complete rotator cuff tear;

FIG. 2A is a front view schematic illustration of a knee-joint;

FIG. 2B is a top view illustration of the knee joint shown in FIG. 2A with the femur removed;

FIG. 2C is a diagrammatic representation of a torn anterior cruciate ligament;

FIG. 3A is a diagrammatic representation of a lyophilization tray according to embodiments of the present disclosure;

FIG. 3B is a diagrammatic representation of a lyophilization tray with adjustable full dividers according to embodiments of the present disclosure;

FIG. 3C is a diagrammatic representation of a lyophilization tray with adjustable partial dividers according to embodiments of the present disclosure;

FIG. 4 is a diagrammatic representation of an implant according to embodiments of the present disclosure in a storage container;

FIG. 5 is a diagrammatic representation of an implant according to embodiments of the present disclosure;

FIG. 6 is a diagrammatic representation of a tissue repair system according to embodiments of the present disclosure utilizing the implant shown in FIG. 4 or 5;

FIG. 7 is a diagrammatic representation of a tissue repair system according to embodiments of the present disclosure utilizing the implant shown in FIG. 4 or 5;

FIG. 8A is a diagrammatic representation of a tissue repair system according to embodiments of the present disclosure utilizing a plurality of the implant shown in FIG. 4 or 5;

FIG. 8B is a diagrammatic representation of a tissue repair system according to embodiments of the present disclosure utilizing a plurality of the implant shown in FIG. 4 or 5;

FIG. 9 is a diagrammatic representation of a tissue repair system according to embodiments of the present disclosure utilizing a plurality of the implant shown in FIG. 4 or 5;

FIG. 10A is a diagrammatic representation of a delivery device suitable for dispensing an implant according to embodiments of the present disclosure;

FIG. 10B is a diagrammatic representation of a delivery device suitable for dispensing an implant according to embodiments of the present disclosure;

FIG. 10C is a diagrammatic representation of a delivery device suitable for dispensing an implant according to embodiments of the present disclosure;

FIG. 10D is a diagrammatic representation of a delivery device suitable for dispensing an implant according to embodiments of the present disclosure;

FIG. 10E is a diagrammatic representation of a delivery device suitable for dispensing an implant according to embodiments of the present disclosure;

FIG. 10F is a diagrammatic representation of a delivery device suitable for dispensing an implant according to embodiments of the present disclosure;

FIG. 11 is a schematic depicting a tissue repair system as shown in FIGS. 6-10F being inserted into a repair site of a rotator cuff tendon;

FIG. 12 is a schematic depicting the tissue repair system as shown in FIGS. 6-10F being inserted into a repair site of an ACL or meniscus;

FIG. 13A is a schematic depicting a tissue repair site prepared to receive an implant as shown in FIGS. 4-5 via a tissue repair system as shown in FIGS. 6-8;

FIG. 13B is a schematic depicting an implant as shown in FIGS. 4-5 being prepared for injection into a tissue repair site; and

FIG. 13C is a schematic depicting an implant as shown in FIGS. 4-5 after injection into a tissue repair site being secured in the repair site.

FIG. 13D is a schematic depicting an implant as shown in FIGS. 4-5 secured in the tissue repair site.

### DEFINITIONS

As used herein, the singular forms “a,” “an,” and “the” include plural references unless the context clearly indicates otherwise. Thus, for example, reference to “an active agent” includes a single active agent as well as a mixture of two or more different active agents or a pharmaceutically acceptable salt, solvate, crystalline form, derivative, prodrug or analogue thereof; and reference to an “excipient” includes a single excipient as well as a mixture of two or more different excipients, and the like.

As used herein, the term “about” in connection with a measured quantity or time, refers to the normal variations in that measured quantity or time, as expected by one of ordinary skill in the art in making the measurement and exercising a level of care commensurate with the objective of measurement. In certain embodiments, the term “about” includes the recited number±10%, such that “about 10” would include from 9 to 11, or “about 1 hour” would include from 54 minutes to 66 minutes.

The term “at least about” in connection with a measured quantity refers to the normal variations in the measured quantity, as expected by one of ordinary skill in the art in making the measurement and exercising a level of care commensurate with the objective of measurement and precisions of the measuring equipment and any quantities higher than that. In certain embodiments, the term “at least about” includes the recited number minus 10% and any quantity that is higher such that “at least about 10” would include 9 and anything greater than 9. This term can also be expressed as “about 10 or more.” Similarly, the term “less than about” typically includes the recited number plus 10% and any quantity that is lower such that “less than about 10” would include 11 and anything less than 11. This term can also be expressed as “about 10 or less.” [0025] Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illustrate certain materials and methods and does not pose a limitation on scope.

The terms “autologous” and “autograft” refer to tissue or cells which originate with or are derived from the recipient, whereas the terms “allogeneic” and “allograft” refer to cells and tissue which originate with or are derived from a donor of the same species as the recipient.

As used herein, the term “active agent” refers to any material that is intended to produce a therapeutic, prophylactic, or other intended effect, whether or not approved by a government agency for that purpose. This term with respect to a specific agent includes the pharmaceutically active agent, and all pharmaceutically acceptable salts, solvates, crystalline forms, derivatives, prodrugs or analogues thereof, where the salts, solvates, crystalline forms, derivatives, prodrugs or analogues are pharmaceutically active.

The term “pharmaceutically acceptable salt” as used herein refers to one or more salts of an active pharmaceutical ingredient (API). For example, pharmaceutically acceptable salts of basic APIs include hydrochloride, mesylate, hydrobromide, acetate and/or fumarate. Pharmaceutically acceptable salts for acidic APIs include sodium, calcium and/or potassium. A pharmaceutically acceptable salt may be chosen for a particular modified release fill formulation based on its aqueous solubility, stability, toxicity, absorption, manufacturability and/or other physicochemical and/or biological considerations.

The term “polymorphism” refers to the ability of an active ingredient (AI) to exist in more than one crystalline form and the term “polymorph” refers to at least one of the crystalline forms of an AI.

As used herein, the terms “therapeutically effective” and an “effective amount” refer to that amount of an active agent, or the rate at which it is administered, needed to produce a desired therapeutic result.

The term “subject” refers to a human or animal, that has demonstrated a clinical manifestation of a condition. The term “subject” may include a person or animal (e.g., a canine) that is a patient being appropriately treated by a medical caregiver for a condition. In the present disclosure, a subject includes, but is not limited to, any mammal, such as human, non-human primate, mouse, rat, dog, cat, horse or cow.

The terms “treatment of” and “treating” include the administration of an active agent(s) with the intent to lessen the severity of a condition and/or a symptom.

The terms “prevention of” and “preventing” include the avoidance of the onset of a condition by a prophylactic administration of the active agent.

The terms “ambient” or “ambient conditions” as used herein refer to temperatures of about 15° C. to less than 37° C. at one (1) atmosphere of pressure.

The term “elevated temperature” as used herein refers to temperatures of about 37° C. and above at one (1) atmosphere of pressure.

The terms “flow,” “flowable,” “flowable fluid,” or “flowable liquid” as used herein refer to a fluid (e.g., a liquid) that has a viscosity of 100,000 cP or less at a temperature of 40° C. or 60° C. when the viscosity is determined using a Thermo Fischer Scientific HAAKE RheoStress 6000 parallel plate with shear stress at 10 Pa and an oscillation frequency of 1 Hz, at 40° C. or 60° C.

The term “particles” as used herein refers to granules, extrudates, powders, pellets, multi-particulates (e.g., coated sub-units, bi-layer sub-units, multi-layer sub-units), minitablets, microcapsules, cuboids, hexoids, rhomboids, spheres, microspheres, cylinders, ovals, fibers, and/or combinations thereof.

The term “w/w %” or “weight concentration” as used herein refers to the mass of the solute as a percentage of the total mass of the solution.

### DETAILED DESCRIPTION OF ILLUSTRATIVE EMBODIMENTS

Reference will now be made in detail to the various embodiments of the present disclosure illustrated in the accompanying drawings. Wherever possible, the same or like reference numbers will be used throughout the drawings to refer to the same or like features. It should be noted that the drawings are in simplified form and are not drawn to precise scale. In reference to the disclosure herein, for purposes of convenience and clarity only, directional terms such as top, bottom, left, right, above, below and diagonal, are used with respect to the accompanying drawings. Such directional terms used in conjunction with the following description of the drawings should not be construed to limit the scope of the present disclosure in any manner not explicitly set forth.

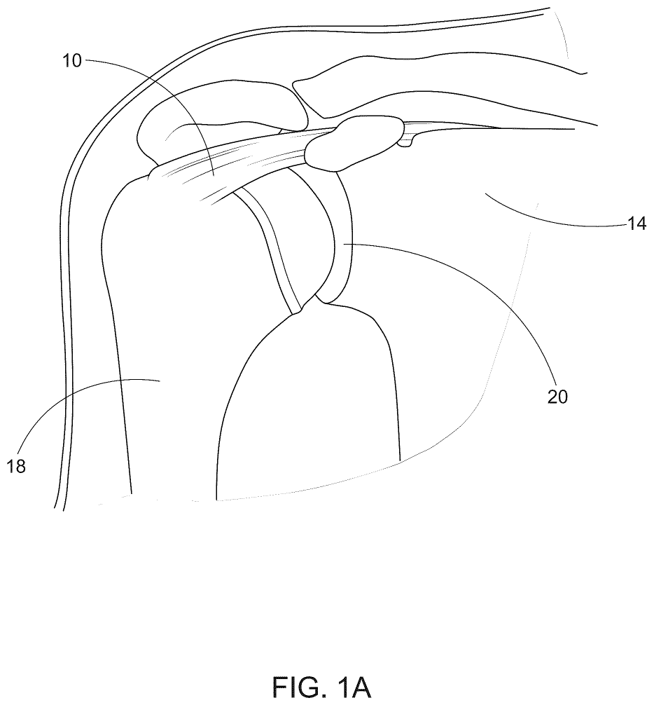

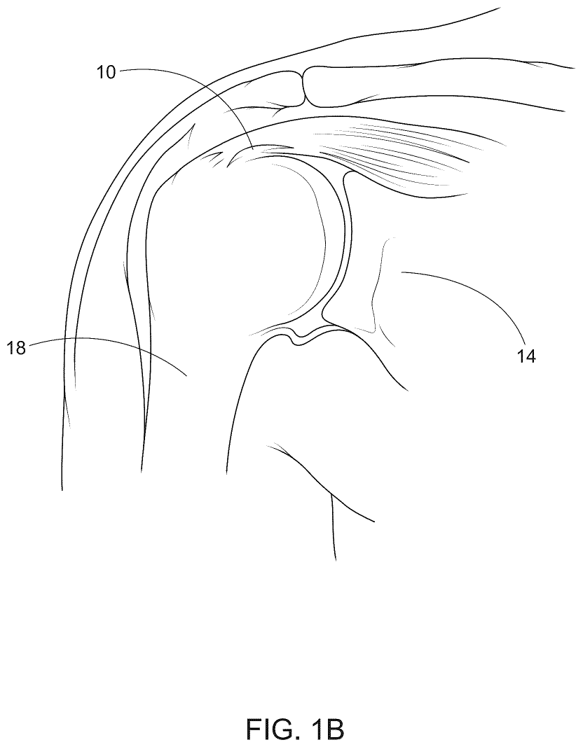

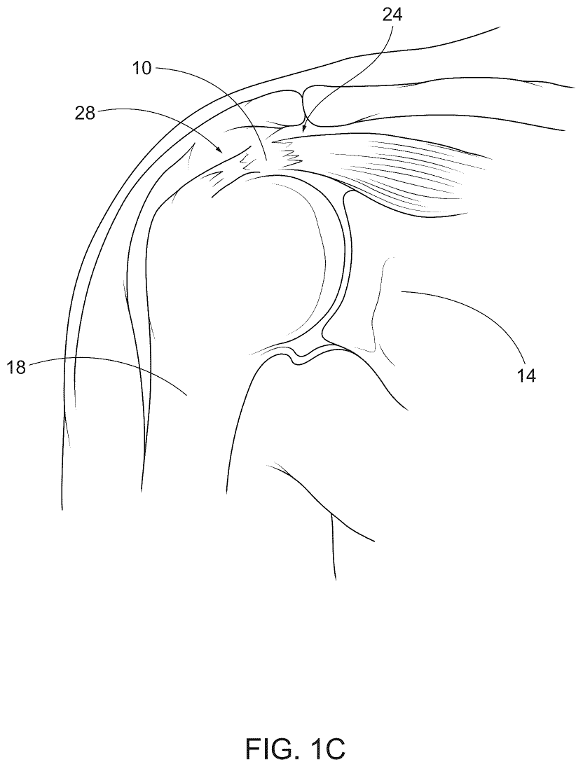

Referring to FIGS. 1A-13D, aspects of the invention relate to compositions, methods of preparation thereof, systems, and methods for repairing tissue. Referring to FIGS. 1A-1C, in one embodiment, the injury may be a torn tendon, for example, a torn rotator cuff. Referring to FIG. 1A, the rotator cuff tendon 10 is a group of muscles and tendons that connects the scapula 14 to the humerus 18. The rotator cuff is comprised of the Supraspinatus, Infraspinatus, Subscapularis and theres minor. The function of the rotator cuff is to provide stability to the glenohumeral joint by compressing the humeral head against the glenoid 20.

Referring to FIGS. 1B-1C, a torn rotator cuff can manifest as a number of different types of tears. The extent and shape of the tear can vary. As shown in FIG. 1B, a partial rotator cuff tear has a partial thickness in depth yet the rotator cuff still remains attached. As shown in FIG. 1C, on the other hand, a complete rotator cuff tear involves a full thickness tear, meaning that the tear has penetrated completely from the top to the bottom of the rotator cuff and no longer connecting the scapula 14 to the humerus 18.

The rotator cuff 10 is ruptured such that it no longer forms a connection between the scapula 14 and the humerus 18. The resulting ends of the ruptured rotator cuff 10 may be of any length. The ends may be of a similar length, or one end may be longer in length than the other. The end on the scapula 14 includes the scapula stump 24. The end on the humerus includes a humeral stump 28. The total length of the supraspinatus is considered to be the length of tissue from humeral footprint to the scapular footprint along a linear axis.

During surgical rotator cuff repair, a repair system is used to place an implant on or over the injury site to bridge the tear in the rotator cuff. The surgical approach may be open, mini-open or arthroscopic, each with its own variation in surgical technique. Current known techniques for repair still present issues with implant deployment and placement, due to confined space, especially in mini-op and arthroscopic repair.

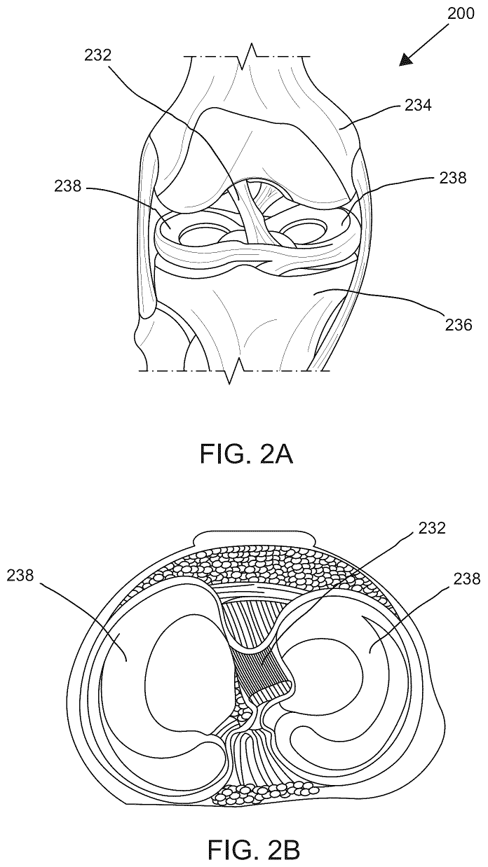

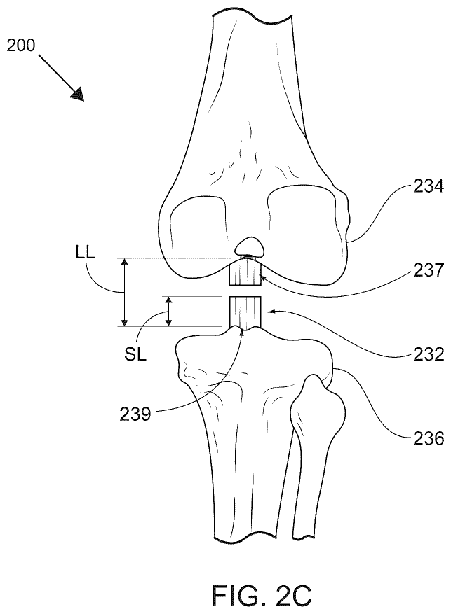

Referring to FIGS. 2A-2C, in another embodiment, the injury may be a torn ligament or a ruptured ligament. A torn ligament may be a partial tear. A torn ligament may also refer to a complete tear. A partial tear is one where a portion of the ligament is damaged, but the ligament remains connected. The tear may be of any length or shape. A ruptured ligament, also known as a complete tear, is one where the ligament has been completely severed providing two separate ends of the ligament. A ruptured ligament may provide two ligament ends of similar or different lengths. The rupture may be such that a ligament stump is formed at one end. For example, there may be a tibial stump connected to the tibia and a femoral stump connected to the femur.

FIG. 2A shows a front view schematic illustration of a knee-joint 200. FIG. 2B shows a top view illustration of the knee joint 200 shown in FIG. 2A with the femur 234 removed. An example of a ruptured anterior cruciate ligament (ACL) 232 is depicted in FIG. 2C. The ACL 232 is one of four strong ligaments that connects the bones of the knee joint 200. The function of the ACL 200 is to provide stability to the knee and minimize stress across the knee joint 200. It restrains excessive forward movement of the lower leg bone, the tibia 236, in relation to the thigh bone, the femur 234, and limits the rotational movements of the knee.

The meniscus 238A, 238B is a layer of cartilage that sits within the knee joint 200. The primary functions of the meniscus 238A, 238B are to stabilize the knee joint 200 and prevent degeneration of the bony surfaces. Without the meniscus, the bones within the knee joint 200 would rub together and gradually wear. The meniscus 238 acts as a shock absorber and cushion, which distributes weight and reduces friction between the femur 234 and tibia 236.

As shown in FIG. 2C, the ACL 232 is ruptured such that it no longer forms a connection between the femur bone 234 and the tibia bone 236. The resulting ends of the ruptured ACL 232 may be of any length. The ends may be of a similar length, or one end may be longer in length than the other. The end on the femur 234 includes the femoral ACL stump 237. The end on the tibia 236 includes a tibial stump 239.

In some embodiments, the injury is a torn meniscus 238A, 238B. A torn meniscus 238A, 238B can manifest as a number of different types of tears (not shown). The extent and shape of the tear can vary. For example, a radial tear occurs within an avascular zone of the meniscus. A horizontal tear runs along the curved fibers of the meniscus. This type of tear can occur in the vascular portion near the outer edge of the meniscus or be more centrally located. A flap tear occurs when part of the cartilage is folded or “peeled” back, causing it to catch on the joint. A bucket-handle tear occurs in the center of the meniscus. A partial meniscus tear has a partial thickness in depth yet the meniscus still remains attached. On the other hand, a complete meniscus tear involves a full thickness tear, meaning that the tear has penetrated completely from the top to the bottom of the meniscus.

The collagen implants, methods of preparation, and methods of implantation thereof according to embodiments herein are suitable to repair tissues as shown, for example, in FIGS. 1A-2C. Suitable tissues include, but are not limited to the rotator cuff, meniscus, ACL, and Achilles.

### Collagen Slurry Compositions

Injectable collagen implants according to various embodiments herein are prepared using collagen slurry compositions. Such slurry compositions may be sterile with collagen solubilized therein, for example, enzyme solubilized collagen. The collagen may be Type I, II, III, IV, V, or X collagen, or combinations thereof. Type I collagen is predominantly found in bone, skin (e.g., in sheet-like structures), and tendon (e.g., in rope-like structures). Type I collagen is densely packed and provides structure to skin, bones, tendons and ligaments. Type I collagen is further typified by its reaction with the protein core of another connective tissue component known as a proteoglycan. Signaling regions that facilitate cell migration are present in Type I collagen. Type II collagen is found in elastic cartilage and provides joint support. Type III collagen may be found in the skin's middle layer (i.e., the dermis), muscles, and blood vessels. Type IV collagen is a thin layer of tissue that supports cells in the kidneys, lungs, intestines, and eyes. Type V collagen may be found in hair and cell surfaces. Type X collagen is found in bones and cartilage and is suitable to support the regulation of matrix mineralization, cartilage development, and tissue remodeling during growth and following injuries.

In embodiments, the collagen may be heterotrimeric, tropocollagen, atelocollagen, fibrillar collagen, or combinations thereof. In some embodiments, the collagen is present in various forms. For example, the collagen may be tropocollagen (e.g., homotrimeric collagen or heterotrimeric collagen), atelocollagen, fibrillar collagen, or combinations thereof. Homotrimeric collagen is comprised of collagen molecules having three identical polypeptide chains. Heterotrimeric collagen also is comprised of collagen molecules having three polypeptide chains, but at least two of the chains differ. Atelocollagen is comprised of collagen molecules that have been purified to remove telopeptide regions known to cause an immune response. Fibrillar collagen is comprised of three chain polypeptide helixes assembled into fibrils; fibrillar collagens aide in the development, growth and maintenance of tissues and organs.

In one or more embodiments, the collagen is Type I, heterotrimeric collagen having two α1(I) chains, and one α2(I) chain. The α1(I) chains may be at least about 100 nm to about 500 nm long, or about 100 nm to about 500 nm long, about 200 nm to about 400 nm long, about 300 nm to about 350 nm long, or any individual value or sub-range within these ranges. In some embodiments, the collagen may comprise tropocollagen and/or atelocollagen and is free of fibrillar collagen in order to reduce the antigenicity of the material.

The collagen may be derived from a source tissue of any mammal species. For example, the collagen may be derived from rat, pig (porcine), cow (bovine), or human tissue. Tendons, ligaments, muscle, fascia, skin, cartilage, tail, or any source of soft collagenous tissue are useful. In various embodiments, the collagen is derived from a bovine tissue (e.g., bovine knee tendons and ligaments, bovine cadaveric knee tissue, bovine elbow tendons and ligaments, etc.). In some embodiments, the collagen is obtained from autologous cells. For example, the collagen may be derived from a patient's cultured fibroblasts. The collagen may then be used in that patient or other patients.

In various embodiments, the collagen is not cross-linked. Although cross-linking may improve mechanical strength of the implant materials, some cross-linking agents may cause cytotoxicity (e.g., glutaraldehyde) and/or foreign body reactions. Calcification and/or biodegradation also may occur with the use of glutaraldehyde. In some embodiments, bone regeneration is impacted with the use of some cross-linked collagen membranes as compared to non-cross-linked counterparts. Cell adhesion and proliferation may be altered by cross-linking, which may impact biocompatibility.

Collagen slurry implant compositions according to embodiments herein may comprise collagen in an amount of about 1 mg/ml to about 1,000 mg/ml of the total volume of the solution, about 5 mg/ml to about 500 mg/ml of the total volume of the solution, about 45 mg/ml to about 300 mg/ml of the total volume of the solution, about 1 mg/ml to less than about 50 mg/ml of the total volume of the solution, or any individual value or sub-range within these ranges. In embodiments, the collagen may be in an amount of about 40 w/w % to about 70 w/w % of the total weight of the collagen slurry, or any individual value or sub-range within this range. The concentration of collagen in the slurry may be determined using a SIRCOL™ assay or similar assay suitable for measuring collagen content.

Collagen slurry implant compositions as disclosed herein may further include a buffer. The buffer may be used to bring the pH of the slurry composition to a neutral level. For example, a buffer with a pK of about 6 to about 8, or any individual value or sub-range therein, may be used to bring the pH of the solution to the desired range for implantation or combination with cells or proteins. In some embodiments, an acid or a base are used to bring the pH of the collagen slurry to about 6 to about 8, or any individual value or sub-range within this range.

According to various embodiments, the collagen slurry contains a buffer in an amount of about 15 w/w % to about 30 w/w %, or any individual value or sub-range within this range. Suitable buffers for use in collagen slurries according to embodiments herein include, but are not limited to, 4-(2-hydroxyethyl)-1-piperazineethanesulfonic acid (HEPES), tris(hydroxymethyl) aminomethane) (TRIS), (3-(N-morpholino)propanesulfonic acid) (MOPS), 3-[N-Tris(hydroxymethyl)methylamino]-2-hydroxypropanesulfonic acid (TAPSO), cacodylate SSC, succinic acid, glycine, sodium phosphate, sodium hydroxide, sodium chloride, magnesium chloride, acetic acid, thrombin buffer, L-ascorbic acid phosphate magnesium salt n-hydrate, MMP-1, MMP-2, MMP-3, MMP-9, RNAase/DNAase, elastase, papain, citrate, sodium citrate, phosphate, saline, or combinations thereof.

In one or more embodiments, the buffer is a zwitterionic buffer. Zwitterionic buffers contain zwitterions, which are molecules having both positive and negative charges enabling the buffer to have both acidic and basic properties. Such buffers have a pKA in the range of about 6 to about 8. Suitable zwitterionic buffers include, but are not limited to, 4-(2-hydroxyethyl)-1-piperazineethanesulfonic acid (HEPES), betaine, tris(hydroxymethyl)aminomethane) (TRIS), (3-(N-morpholino)propanesulfonic acid) (MOPS), (3-(Cyclohexylamino)-1-propanesulfonic acid) (CAPS), (2-(N-morpholino) ethanesulfonic acid) (MES), 2,2′-[(2-amino-2-oxoethyl)azanediyl]diacetic acid (ADS), 2,2′-(Piperazine-1,4-diyl)di(ethane-1-sulfonic acid) (PIPES), 2-[(2-Amino-2-oxoethyl)amino]ethane-1-sulfonic acid (ACES), 2-Hydroxy-3-(morpholin-4-yl)propane-1-sulfonic acid (MOPSO), cholamine chloride hydrochloride, N,N-Bis(2-hydroxyethyl)taurine (BES), 2-{[1,3-Dihydroxy-2-(hydroxymethyl)propan-2-yl]amino}ethane-1-sulfonic acid (TES), 3-(N,N-Bis(2-Hydroxyethyl)amino)(DIPSO), 3-[[1,3-dihydroxy-2-(hydroxymethyl)propan-2-yl]amino]-2-hydroxypropane-1-sulfonic acid (TAPSO), acetamido-glycine, Piperazine-1,4-bis(2-hydroxy-3-propanesulfonic acid),dihydrate (POPSO), N-(2-Hydroxyethyl)piperazine-N′-(3-propanesulfonic acid) (HEPPSO), 3-[4-(2-Hydroxyethyl) piperazin-1-yl]propane-1-sulfonic acid (HEPPS), N-[1,3-Dihydroxy-2-(hydroxymethyl)propan-2-yl]glycine (Tricine), glycinamide, glycylglycine, bicine, 3-{[1,3-Dihydroxy-2-(hydroxymethyl) propan-2-yl]amino}propane-1-sulfonic acid (TAPS), or combinations thereof.

The buffer may include a single component, or may have multiple components. If the buffer has a single component, the buffer should have properties that enable it to produce a solution having a desirable pH range and osmolarity. In some instances, it is desirable to have at least two buffer components, for example, a collagen buffer solution and a neutralizing buffer. The collagen buffer solution may be used to prepare the collagen in a solution. In some instances, the prepared collagen solution may be stored for extended periods of time. In some embodiments, the buffer comprises TRIS.

Collagen slurries according to embodiments herein may further include one or more salts. Some salts are beneficial to the healing process and control infection when the collagen implant, formed from the collagen slurry, is implanted and biodegrades within the subject. Salts also may be used to produce desirable properties in the implant such as porosity, surface attraction, biomechanical support, biocompatibility, and/or antimicrobial properties. Salts that may be incorporated into the collagen slurry include, but are not limited to, calcium containing salts, magnesium containing salts, sodium containing salts, calcium chloride, magnesium chloride, sodium chloride, or combinations thereof. In some embodiments, the salt comprises sodium. The collagen slurry may contain one or more salt in an amount of about 2 w/w % to about 5 w/w %, or any individual value or sub-range within this range.

Collagen slurries as described herein may further include one or more electrolytes. Electrolytes play a role in surface modification of the collagen implant to promote bone integration and/or improve biocompatibility. The composition of the electrolyte in collagen implants made from the described collagen slurries can modify the properties of the implant surface to reduce inflammation and promote healing. Suitable electrolytes for collagen slurry compositions include, but are not limited to chloride, nitrate, sulfate, silicate, phosphate, aluminate, sodium tetraborate, or combinations thereof. In some embodiments, the electrolyte comprises chloride. The one or more electrolyte may be present in the collagen slurry in an amount of about 5 w/w % to about 11 w/w %, or any individual value or sub-range within this range.

In various embodiments, the collagen slurries contain one or more glycosaminoglycans (GAGs). GAGs are long chain polysaccharides that promote implant biocompatibility and tissue integration. Suitable GAGs for implants configured for injection according to embodiments herein include, but are not limited to heparan sulfate, chondroitin sulfate, dermatan sulfate, hyaluronic acid, keratan sulfate, or combinations thereof. GAGs may be present in the implants according to embodiments herein in an amount of less than about 5 w/w %, less than about 2 w/w %, less than about 1 w/w %, or about 0.001 w/w % to less than about 2 w/w %, or any individual value or sub-range within these ranges. In some embodiments, each particle of the plurality of particles includes one or more GAG in an amount of greater than 100 μg/g, or about 100 μg/g to about 1,000 μg/g, or any individual value or sub-range within these ranges.

According to various embodiments, collagen slurries are combined with cells such as platelets, white blood cells, red blood cells, stem cells, fibroblasts, or combinations thereof. In some embodiments, the cells are derived from the subject to be treated. In other embodiments, the cells are derived from a donor that is allogeneic to the subject.

In certain embodiments, platelets may be obtained as platelet rich plasma (PRP). PRP contains fibrin and platelets together with other plasma proteins found in the blood. PRP may further include white blood cells and red blood cells found in this preparation. In some embodiments, the PRP has a platelet concentration at least about 100 K/ml, at least about 300 K/ml, about 100 K/ml to about 10,000 K/ml, or any individual value or sub-range within these ranges. In embodiments, the platelet concentration is about 1.0 times to about 5.0 times the platelet concentration in the blood of the patient, or any individual value or sub-range within this range. In order to maintain the stability of the cells, a physiologic pH (i.e., about 6.2 to about 7.6) and a physiologic plasma osmolarity (i.e., about 280 osms/kg about 360 osms/kg) is used. In order to enhance the function of the PRP, PRP may be used within seven (7) days of being drawn from the patient or donor. The PRP may be isolated from the patient at time of surgery. It may be stored at about 20° C. to about 37° C. (room temp to body temp). However, isolation and storage of the cells may be achieved by any methods and for any length of time known in the art for maintaining the activity of the active components.

In a non-limiting example, platelets may be isolated from a subject's blood using techniques known to those of ordinary skill in the art. As an example, a blood sample may be drawn into a tube containing an anticoagulant, and the subsequent solution centrifuged at 700 rpm for 20 minutes and the platelet-rich plasma upper layer removed. Platelet density may be determined using a cell count as known to those of ordinary skill in the art. The platelet rich plasma may be mixed with collagen and applied to the patient.

In a non-limiting example, white blood cells may be isolated from a subject's blood using techniques known to those of ordinary skill in the art. As an example, a blood sample may be drawn into a tube containing an anticoagulant and centrifuged at 700 rpm for 20 minutes and the buffy coat containing white blood cells removed. The white blood cell density may be determined using a cell count as known to those of ordinary skill in the art. The white blood cells can be mixed with collagen and applied to the patient.

Mixing collagen and/or a collagen implant composition as described herein with blood, platelet rich plasma, white blood cells, etc. may form a viscous paste or putty mixture, depending on the ratio. Suitable volumetric ratios include, but are not limited to about 1:1 to about 100:1 of the volume of the collagen or collagen implant composition to the volume of the blood, platelet rich plasma, white blood cells, etc., or any individual value or sub-range within this range. When collagen or a collagen implant composition in an amount of about 3 ml to about 1,000 ml, or any individual value or sub-range therein, is mixed with a volume of about 3 ml to about 10 ml of blood, platelet rich plasma, white blood cells, etc., the resulting mixture may be viscous enough to be expelled from the delivery device, but stable enough to stay within the confines of the anatomical fields into which it is injected. Suitable viscosities for the collagen, collagen composition, or for the mixture with blood, platelet rich plasma, white blood cells, etc., include, but are not limited to, about 1 cP to about 10 cP, about 1.2 cP to about 8 cP, about 3.5 cP to about 5.5 cP, or any individual value or sub-range within these ranges.

The collagen solution may also include any one or more of an anti-plasmin agent, an extracellular matrix (ECM) protein, other protein or enzyme inhibitors, antibodies to plasmin, antibodies to tissue plasminogen activator or urokinase plasminogen activator, non-toxic crosslinkers, calcium, dextrose or other sugars and cell nutrients in physiological concentrations. Anti-plasmin agents include but are not limited to antifibrinolytic enzymes such as plasminogen inactivator, plasminogen binding α₂ antiplasmin, non-plasminogen binding α₂ antiplasmin, α₂ macroglobulin, α₂ plasmin inhibitor, a₂ antiplasmin, and thrombin activatable fibrinolysis inhibitor. Other protein or enzyme inhibitors include but are not limited to anti-enzymatic proteins including inhibitors of collagenase, trypsin, matrix metalloproteinases, elastase and hyaluronidase. The ECM is composed of fibrillar and non-fibrillar components. The major fibrillar proteins are collagen and elastin. The ECM includes for instance, diverse combinations of collagens, fibrinogen, proteoglycans, elastin, hyaluronic acid, and various glycoproteins including laminin, fibronectin, heparan sulfate proteoglycan, and entactin. Non-toxic crosslinkers include but are not limited to tissue transglutaminases, lysyl oxidase, fibrin, fibronectin, and reducible and non-reducible crosslink precursor molecules.

Collagen slurries according to embodiments herein may include one or more additional components, such as insoluble collagen, other extracellular matrix proteins (ECM), such as proteoglycans and glycosaminoglycans, fibronectin, laminin, entectin, decorin, lysyl oxidase, crosslinking precursors (reducible and non-reducible), elastin, elastin crosslink precursors, cell components such as, cell membrane proteins, mitochondrial proteins, nuclear proteins, cytosomal proteins, and cell surface receptors, growth factors, such as, PDGF, TGF, EGF, and VEGF, hydroxyproline, or combinations thereof. In one or more embodiments, the collagen slurries comprise one or more peptide, proteinase inhibitor, collagenase inhibitor, zinc ions, retinol, vitamin, steroid, hormone, cytokine, clotting factor, angiogenic protein, antiangiogenic protein, anti-protease protein, bone morphogenic protein, osteoinductive factor, fibronectin, cementum attachment extract, ketanserin, interlenkin-1, human alpha thrombin, an anti-inflammatory, or combinations thereof. According to various embodiments the anti-protease protein may include, but is not limited to alphalantitrypsin, the antiangiogenic protein comprises endostatin, angiostatin, or combinations thereof. In one or more embodiments, the growth factor comprises endothelial cell growth factor, epidermal growth factor, transforming growth factor-beta, insulin-like growth factor, platelet derived growth factor, periodontal ligament chemotactic factor, vascular endothelial growth factor, fibroblast growth factor, or combinations thereof. In embodiments, the hormone comprises human growth hormone, animal growth hormones, or combinations thereof. In at least one embodiment, the vitamin comprises vitamin A, vitamin B6, vitamin B9, vitamin B12, vitamin C, vitamin D, vitamin K, or combinations thereof.

In various embodiments, the anti-inflammatory includes a non-steroidal anti-inflammatory drug (NSAID), acetaminophen, an opioid, a cannabinoid, salicylic acid, lidocaine, or combinations thereof. Suitable NSAIDs include, but are not limited to ibuprofen, naproxen, diclofenac, celecoxib, mefenamic acid, etoricoxib, indomethacin, or combinations thereof. In some embodiments, the opioid comprises hydrocodone, oxycodone, morphine, oxymorphone, codeine, or combinations thereof. Suitable cannabinoids include, but are not limited to, tetrahydrocannabinol (THC), cannabidiol (CBD) and cannabinol (CBN). Other cannabinoids include for example, cannabichromene (CBC), cannabigerol (CBG) cannabinidiol (CBND), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM). As used herein THC, CBD, CBN, CBC, CBG, CBND, CBL, CBV, THCV, CBDV, CBCV, CBGV and CBGM refer to the decarboxylated form of the cannabinoid. Whereas THCA, CBDA, CBNA, CBCA, CBGA, CBNDA, CBLA, CBVA, THCVA, CBDVA, CBCVA, CBGVA and CBGAM (cannabigerolic acid monomethyl ether) refer to the corresponding acid form of the cannabinoid.

According to various embodiments, collagen slurries as described herein comprise collagen in an amount of about 40 w/w % to about 70 w/w %, a buffer (as described herein) in an amount of about 15 w/w % to about 30 w/w %, a first salt in an amount of about 2 w/w % to about 5 w/w %, a second salt in an amount of about 2 w/w % to about 5 w/w %, an electrolyte in an amount of about 5 w/w % to about 11 w/w %, and glycosaminoglycan in an amount of less than about 2 w/w %, or about 0.001 w/w % to less than about 2 w/w %, or any individual value or sub-range within these ranges. In some embodiments, the buffer comprises HEPES. In one or more embodiments, the first and second salt are independently chosen from calcium and sodium. In various embodiments, the electrolyte may be chloride.

In various embodiments, collagen slurry compositions have a collagen content of greater than 400 mg/g, about 400 mg/g to about 10,000 mg/g, or any individual value or sub-range within these ranges. Collagen slurry compositions according to embodiments herein may include DNA in an amount of less than 50,000 ng/g, about 5 ng/g to less than about 50,000 ng/g, or any individual value or sub-rang within these ranges. Collagen slurry compositions may include one or more GAGs in an amount of greater than 100 μg/g, greater than about 100 μg/g to about 8,000 μg/g, or any individual value or sub-range within these ranges. Collagen slurry compositions may include a phospholipid having a phospholipid count of less than 3,000 μM/g, about 1 μM/g to less than about 3,000 μM/g, or any individual value or sub-range within these ranges. In some embodiments, collagen slurry composition include pepsin in an amount of less than 12.5 mg/g, about 0.1 mg/g to less than about 12.5 mg/g, or any individual value or sub-range within these ranges. In some embodiments, the collagen slurry composition comprises a collagen content of greater than 400 mg/g, a GAG content of greater than 100 μg/g, a DNA content of less than 50,000 ng/g, a phospholipid count of less than 3,000 μM/g and a pepsin content of less than 12.5 mg/g.

### Methods of Preparing Collagen Slurry Compositions

Further described herein are methods of preparing collagen slurry compositions. According to at least one embodiment, the methods of preparing the collagen slurry include preparing a source tissue prior to performing a salt extraction. Preparing the source tissue may include obtaining one or more cadaveric joints (e.g., knees) from a mammal (e.g., human, bovine, rat, porcine, etc.), contacting the one or more cadaveric joints with a solution to reduce bacterial load on the skin, incising the skin and separating underlying layers to expose target tissue, and harvesting the target tissue. In some embodiments, preparing the source tissue comprises lyophilizing harvested target tissue form one or more cadaveric knees of a mammal to form a lyophilized tissue. Methods of preparing a collagen slurry may include preparing ground tissue by grinding freeze-dried collagenous tissue that has been isolated from the mammal. This can be performed by a homogenizer as described herein or by a similar device. A similar device is one that is useful for breaking the tissue into small pieces that can be effectively extracted.

If obtained frozen, the joints are thawed before harvesting the desired tissues. The knees may be prepared with solutions to reduce the bacterial load on the skin. Then the skin is incised, and the underlying layers split until the desired tissue is exposed. The tissue may be harvested and placed into sterile containers. The collagen may be isolated from the tissue source and mechanically minced and extracted. In various embodiments, the collagen is kept cold (4′C or on ice) during storage and throughout parts of the preparation. After harvesting, the tissue may be frozen at −20° C. or −80′C and stored for up to one year. Alternatively, the tissue may be lyophilized immediately.

Following lyophilization and/or storage, the tissue may be homogenized using any suitable device for cutting tissue including, but not limited to, a blender, homogenizer, food processor, scalpel or a combination thereof. The small pieces of tissue are placed into a sterile salt solution which may contain antibiotics and/or an antimycotic. Homogenization may be carried out at room temperature, a temperature below room temperature, at about 4° C. to less than room temperature, below 4° C., or any individual value or sub-range within these ranges. In one embodiment, dry ice is used to maintain the desired temperature of the tissue. In some embodiments, the homogenizing instrument, vessel, parts, or combinations thereof are cooled to maintain the desired temperature. In various embodiments, one or more parts of the homogenizing instruments are sterilized. In embodiments, all instrumentation which may contact the tissue are sterilized. In embodiments, all instruments that may contact the tissue are substantially free of endotoxin.

In at least one embodiment, methods for preparing a collagen slurry include performing a salt extraction of a source tissue to produce a salt extracted collagen. The source tissue may be from any mammal species. For example, the source tissue may be human, bovine, porcine, or combinations thereof. In some embodiments, the source tissue is harvested from any site yielding connective tissue.

The salt extraction may be used to treat the tissue. The homogenized tissue may be placed into a tube containing a salt solution at a concentration of about 10% to about 30% by weight, greater than 30% by weight, less than 10% by weight, or any individual value or sub-range within these ranges. According to various embodiments, the salt solution comprises sodium chloride (NaCl), calcium chloride (CaCl₂), or combinations thereof. The salt extraction may be carried out at room temperature, a temperature below room temperature, at about 4° C. to less than room temperature, below 4° C., or any individual value or sub-range within these ranges.

In at least one embodiment, the methods further include performing a first salt extraction on the ground tissue to produce a salt extracted collagen, treating the salt extracted collagen with a detergent solution, followed by an enzyme digestion (e.g., using elastase, in some embodiments RNase and/or DNase, in some embodiments trypsin, papain or one or more collagenase solutions) and ethylenediaminetetraacetic acid (EDTA) incubation to produce a collagen mixture, performing a second salt extraction on the collagen mixture and centrifuging the salt extracted mixture to produce a pellet, incubating the pellet with a citrate buffer, followed by acid solubilization and pepsin digestion.

Performing the salt extraction may include contacting the source tissue with sodium chloride or calcium chloride. In embodiments, the source tissue is contacted with sodium chloride in a water or buffer solution at a concentration of at least about 1 M, or about 1 M to about 7 M, or any individual value or sub-range within these ranges. The buffer solution may be at physiological pH, for example, a pH of about 5 to about 9, or about 6 to about 7, or any individual value or sub-range within these ranges. In some embodiments, the buffer is HEPES, TRIS, MOPS, TAPSO, cacodylate SSC, succinic acid, glycine, sodium phosphate, sodium hydroxide, sodium chloride, magnesium chloride, acetic acid, thrombin buffer, L-ascorbic acid phosphate magnesium salt n-hydrate, MMP-1, MMP-2, MMP-3, MMP-9, RNAase/DNAase, elastase, papain, citrate, sodium citrate, phosphate, saline, or combinations thereof.

A treatment to remove cells and cell debris may be carried out. Agents suitable to remove cell debris include, but are not limited to, enzymes, chemicals, detergents, salt solutions, hyperosmotic solutions, or combinations thereof. A physical agent, such as ultrasonic agitation, ultrasound, mechanical agitation, electronic stimulation, or combinations thereof may be used as a decellularization agent. The agents used to remove the cells and cell debris may comprise synthetic and/or natural materials. In various embodiments, the decellularization agent may be chosen from an enzyme, sodium dodecyl sulfate (SDS), DNAse, RNAse, Triton X, hypertonic NaCl, elastase, trypsin, a matrix metalloproteinase, or combinations thereof. The tissue may be exposed to these decellularization agents for about 1 hour to about 10 hours, about 1 hour to 5 hours, about 5 hours to about 10 hours, about 10 hours to about 24 hours, greater than about 24 hours, less than about 1 hour, or any individual value or sub-range within these ranges. The tissue may be exposed to these decellularization agents at a specific temperature. In one embodiment, the decellularization process may be carried out at room temperature, a temperature below room temperature, at about 4° C. to less than room temperature, below 4° C., or any individual value or sub-range within these ranges.

According to various embodiments, the tissue may be washed after the decellularization process. The wash may be performed using water, saline, other diluents, or combinations thereof. A second salt extraction may be performed after the decellularization process. A rinse process may be performed after the decellularization process or after the salt extraction.

Additional collagen may be extracted using a buffer as described herein (e.g., a citrate buffer). The buffer may be at a pH of about 4. The buffer may be placed in contact with the tissue for up to about 48 hours. The additional extraction with the buffer may be carried out at room temperature, a temperature below room temperature, at about 4° C. to less than room temperature, below 4° C., or any individual value or sub-range within these ranges. The tissue may be rinsed after addition of the buffer to remove all or some of the buffer.

Ultracentrifugation may be used to process the tissue. Spin speeds of at least about 1000 rpm may be used to pellet the tissue. The ultracentrifugation may be alternated and repeated with wash steps with sterile solutions of acid, base, or neutral solutions.

An additional enzyme step may be used to break the collagen and/or glycosaminoglycans down into smaller fragments. Suitable enzymes include, but are not limited to, collagenase type I, collagenase type II, collagenase type III, matrix metalloproteinase, matrix metalloproteinase-1, matrix metalloproteinase-13, pepsin, elastase, trypsin, aggrecanase, chondroitinase, or combinations thereof.

The collagen materials are sterile for in vivo use. The collagen slurry may be sterilized and/or components of the solution may be isolated under sterile conditions using sterile techniques to produce a sterile composition. The final desired properties of the composition may be determinative of how the solution is sterilized because some sterilization techniques may affect properties such as viscosity. If certain components of the solution are not to be sterilized, i.e., the collagen isolated from natural sources, the remaining components can be combined and sterilized before addition of the collagen, or each component can be sterilized separately. The solution can then be made by mixing each of the sterilized components with the collagen that has been isolated using sterile techniques under sterile conditions. Sterilization may be accomplished, for instance, by autoclaving at temperatures of about 115° C. to 130° C., about 120° C. to 125° C., or any individual value or sub-range within these ranges, for about 30 minutes to about 24 hours, about 45 min to about 12 hours, about 30 minutes to about 1 hour, or any individual value or sub-range within these ranges. In some embodiments, gamma radiation is used for sterilizing components. Filtration also may be employed as may sterilization with ethylene oxide. In some embodiments, sterilization occurs at low temperature conditions such as at room temperature, a temperature below room temperature, at about 4° C. to less than room temperature, below 4° C., about −20° C. to about −30° C., or any individual value or sub-range within these ranges.

In one or more embodiments, methods of preparing a collagen slurry include preparing a neutralized collagen slurry, heating the neutralized collagen slurry and freeze-drying the heated slurry. Preparing the neutralized collagen slurry may be achieved by mixing the collagen slurry with a neutralizing buffer. It may also involve adding a salt (e.g., calcium) containing solution. Heating may be performed, for instance in a mold in a dry oven or in an incubator.

The slurry may then be lyophilized to remove all water and subsequently resuspended with a measured amount of water, saline or other diluent to result in a slurry with the desired concentration of collagen. A strong acid or base may be added to the slurry to bring the pH to a desired level to inactivate any enzymes or chemicals used in the processing of the slurry that are desired to be inactivated before implantation. Additional acid or base, or a buffer with a pK of about 7 to about 8, may be used to bring the pH of the solution to the desired range for implantation or combination with cells or proteins. The osmolarity of the slurry may be adjusted to the desired range using a salt solution, or an acid or base. Once the slurry has the appropriate pH and osmolarity, it may be subjected to heat or cold to cause self-assembly or gelation of the collagen. After gelation, lyophilization of the scaffold may be used to produce an implant, sponge, or powder. Alternatively, the solution may be maintained as a gel. In an embodiment, conditions are maintained to prevent collagen self-assembly until after implantation of the collagen material into the joint.

The methods may further include performing a detergent extraction of the salt extracted collagen to produce a detergent extracted collagen. In various embodiments, performing the detergent extraction comprises contacting the salt extracted collagen with a detergent comprising sodium dodecyl sulphate having about 5 to about 100 ethylene oxide units, polyethylene glycol tert-octylphenyl ether, polyoxyethylene sorbitan monolaurate, 3-[(3-cholamidopropyl) dimethylammonio]-2-hydroxy-1-propanesulfonate, or combinations thereof. Suitable polyoxyethylene sorbitan monolaurates include, but are not limited to polysorbate 20, polysorbate 80, or combinations thereof.

In one or more embodiments, methods for preparing a collagen slurry include performing an enzyme digestion of the detergent extracted collagen to produce an enzyme extracted collagen. Performing enzymatic digestion may include contacting the detergent extracted collagen with deoxyribonuclease, ribonuclease, or combinations thereof. In at least one embodiment, performing enzymatic digestion comprises contacting the detergent extracted collagen with pepsin, collagenase type I, collagenase type II, collagenase type III, matrix metalloproteinase, matrix metalloproteinase-1, matrix metalloproteinase-13, elastase, trypsin, aggrecanase, or chondroitinase. In some embodiments, performing the enzymatic digestion comprises contacting the detergent extracted collagen with pepsin at a pH of about 2 to about 3, or about 1.5 to about 2.5, or any individual value or sub-range within this range.

In at least one embodiment, the methods include performing an acid solubilization of the enzyme extracted collagen to produce a purified collagen slurry. In embodiments, performing the acid solubilization includes solubilizing the enzyme extracted collagen in hydrochloric acid (HCl), sulfuric acid (H₂SO₄), phosphoric acid (H₃PO₄), nitric acid (HNO₃), acetic acid (CH₃COOH), formic acid, maleic acid, oxalic acid, or combinations thereof. In at least one embodiment, the acid solubilization is performed with hydrochloric acid. The concentration of the purified collagen slurry may be adjusted with water or a buffer to about 10 g/L to about 80 g/L, about 20 g/L to about 70 g/L, about 30 g/L to about 60 g/L, or any individual value or sub-range within these ranges. Suitable buffers include, but are not limited to HEPES, TRIS, MOPS, TAPSO, cacodylate SSC, succinic acid, glycine, sodium phosphate, sodium hydroxide, sodium chloride, magnesium chloride, acetic acid, thrombin buffer, L-ascorbic acid phosphate magnesium salt n-hydrate, MMP-1, MMP-2, MMP-3, MMP-9, RNAase/DNAase, elastase, papain, citrate, sodium citrate, phosphate, saline, or combinations thereof.

Methods of preparing the collagen slurry may further include combining the purified collagen slurry with a salt, growth factor, peptide, proteinase inhibitor, collagenase inhibitor, zinc ions, retinol, vitamin, steroid, hormone, cytokine, clotting factor, angiogenic protein, antiangiogenic protein, anti-protease protein, bone morphogenic protein, osteoinductive factor, fibronectin, cementum attachment extract, ketanserin, interlenkin-1, human alpha thrombin, an anti-inflammatory, or combinations thereof.

Suitable salts include, but are not limited to calcium, magnesium, or combinations thereof. In some embodiments, the anti-protease protein comprises alphalantitrypsin. The antiangiogenic protein may include endostatin, angiostatin, or combinations thereof. Suitable growth factors include, but are not limited to endothelial cell growth factor, epidermal growth factor, transforming growth factor-beta, insulin-like growth factor, platelet derived growth factor, periodontal ligament chemotactic factor, vascular endothelial growth factor, fibroblast growth factor, or combinations thereof. The hormone may include human growth hormone(s), animal growth hormone(s), or combinations thereof. Suitable vitamins include, but are not limited to vitamin A, vitamin B6, vitamin B9, vitamin B12, vitamin C, vitamin D, vitamin K, or combinations thereof.

According to various embodiments, the anti-inflammatory includes, but is not limited to a non-steroidal anti-inflammatory drug (NSAID), acetaminophen, an opioid, a cannabinoid, salicylic acid, lidocaine, or combinations thereof. In some embodiments, the NSAID includes ibuprofen, naproxen, diclofenac, celecoxib, mefenamic acid, etoricoxib, indomethacin, or combinations thereof. The opioid may include hydrocodone, oxycodone, morphine, oxymorphone, codeine, or combinations thereof. Suitable cannabinoids include, but are not limited to, THC, CBD, CBN. CBC, CBG, CBND, CBL, CBV, THCV, CBDV, CBCV, CBGV, CBGM, THCA, CBDA, CBNA, CBCA, CBGA, CBNDA, CBLA, CBVA, THCVA, CBDVA, CBCVA, CBGVA, or combinations thereof.

Methods of preparing the collagen slurry may further include neutralizing at least one of the salt extracted collagen, detergent extracted collagen, enzyme extracted collagen, or collagen slurry by rinsing with water or a buffer. In one or more embodiments, the buffer is at physiological pH, for example, a pH of about 5 to about 9, or about 6 to about 7, or any individual value or sub-range within these ranges. Suitable buffers include, but are not limited to HEPES, TRIS, MOPS, TAPSO, cacodylate SSC, succinic acid, glycine, sodium phosphate, sodium hydroxide, sodium chloride, magnesium chloride, acetic acid, thrombin buffer, L-ascorbic acid phosphate magnesium salt n-hydrate, MMP-1, MMP-2, MMP-3, MMP-9, RNAase/DNAase, elastase, papain, citrate, sodium citrate, phosphate, saline, or combinations thereof.

### Injectable Collagen Implants

According to further embodiments, described herein are collagen implants configured for injection into a repair site. In contrast to collagen implants of the prior art, implants as described herein may be injected (e.g., in the form of a liquid, slurry, suspension, etc.) into the repair site at or near a target tissue. The collagen implants may be a liquid, solid, or semi-solid/liquid material useful for implantation into a human subject or animal subject to repair damaged tissue and/or to deliver compounds and/or cells to the subject. The collagen implant may be a compressible and expandable, biodegradable, porous material that has some resistance to degradation by fluids in the tissue repair site, (for example, synovial fluid). The collagen is soluble, e.g., acidic or basic. In some embodiments, the term collagen implant may refer to an injectable fluid, scaffold, and/or sponge. In some embodiments, once injected into the repair site, the solution may harden to form a solid or semi-solid material, and/or are bioabsorbable.

Collagen implants configured for injection as described herein may include a plurality of particles. In some embodiments, at least two particles of the plurality of particles are a different shape and/or size. The plurality of particles may be in the form of a pieces, granules, extrudates, powders, pellets, multi-particulates (e.g., coated sub-units, bi-layer sub-units, multi-layer sub-units), minitablets, microcapsules, cuboids, hexoids, rhomboids, spheres, microspheres, cylinders, ovals, fibers, and/or combinations thereof. Each of the plurality of particles may have a mean size suitable to pass through a luer lock opening of an open bore syringe. The mean size of each of the plurality of particles may be less than about 100 mm, less than about 70 mm, less than about 1 mm, less than about 0.1 mm, less than about 100 μm, less than about 50 μm, less than about 10 μm, less than about 1 μm, about 0.1 μm to about 100 mm, about 2 mm to about 70 mm, about 2 mm to about 25 mm, or any individual value or sub-range within these ranges.

In one or more embodiments, the plurality of particles comprises a porosity of about 50% to about 99%, or about 80% to 98%, or any individual value or sub-range within these ranges. Each of the plurality of particles may have a porosity of less than about 10%, or about 1% to about 10%, or any individual value or sub-range within these ranges. In some embodiments, collagen implants as described herein may have a porosity of about 50% to about 99%, or about 80% to 98%, or any individual value or sub-range within these ranges.

Each of the plurality of particles may be formed of a combination of collagen with other components. For example, each particle of the plurality of particles may include collagen (e.g., sourced/harvested as described herein) in an amount of about 40 w/w % to about 70 w/w %, about 50 w/w % to about 60 w/w %, or any individual value or sub-range within these ranges. The collagen may be derived from a source tissue from any mammal species. For example, the collagen may be derived from human, bovine, or porcine tissues, or combinations thereof. In various embodiments, the collagen is derived from a soft tissue. The collagen may be Type I, Type II, Type III, Type IV, Type V, Type X, or combinations thereof. In embodiments, the collagen is fibrillar collagen, tropocollagen, atelocollagen, heteriotrimeric collagen, or combinations thereof.

Each of the plurality of particles may further include at least one salt in an amount of about 1 w/w % to about 10 w/w %, 2 w/w % to about 5 w/w %, about 3 w/w % to about 4 w/w %, or any individual value or sub-range within these ranges. Suitable salts include, but are not limited to, calcium containing salts, magnesium containing salts, sodium-containing salts, calcium chloride, magnesium chloride, sodium chloride, or combinations thereof. In some embodiments, the salt comprises calcium, magnesium, or combinations thereof.

In various embodiments, each particle of the plurality of particles includes at least one electrolyte in an amount of about 1 w/w % to about 20 w/w %, about 2 w/w % to about 18 w/w %, about 5 w/w % to about 11 w/w %, about 6 w/w % to about 10 w/w %, or any individual value or sub-range within these ranges. Suitable electrolytes include, but are not limited to chloride, nitrate, sulfate, silicate, phosphate, aluminate, sodium tetraborate, or combinations thereof. In some embodiments, the electrolyte comprises chloride.

Each particle of the plurality of particles may further include one or more GAG in an amount of less than about 10 w/w %, less than about 5 w/w %, less than about 2 w/w %, less than about 1 w/w %, about 0.1 w/w % to about 10 w/w %, or about 0.001 w/w % to less than about 2 w/w %, or any individual value or sub-range within these ranges. In some embodiments, each particle of the plurality of particles includes one or more GAG in an amount of greater than 100 μg/g, or about 100 μg/g to about 1,000 μg/g, or any individual value or sub-range within these ranges. Suitable GAGs include, but are not limited to heparan sulfate, chondroitin sulfate, dermatan sulfate, hyaluronic acid, keratan sulfate, or combinations thereof.

In various embodiments, each particle of the plurality of particles further comprises a buffer. In some embodiments, the buffer is a zwitterionic buffer. Suitable buffers include, but are not limited to HEPES, TRIS, MOPS, TAPSO, cacodylate SSC, succinic acid, glycine, sodium phosphate, sodium hydroxide, sodium chloride, magnesium chloride, acetic acid, thrombin buffer, L-ascorbic acid phosphate magnesium salt n-hydrate, MMP-1, MMP-2, MMP-3, MMP-9, RNAase/DNAase, elastase, papain, citrate, sodium citrate, phosphate, saline, or combinations thereof.

In one or more embodiments, each particle of the plurality of particles further includes a growth factor, peptide, proteinase inhibitor, collagenase inhibitor, zinc ions, retinol, vitamin, steroid, hormone, cytokine, clotting factor, angiogenic protein, antiangiogenic protein, anti-protease protein, bone morphogenic protein, osteoinductive factor, fibronectin, cementum attachment extract, ketanserin, interleukin-1, human alpha thrombin, an anti-inflammatory, or combinations thereof. The anti-protease protein may include alphalantitrypsin, the antiangiogenic protein comprises endostatin, angiostatin, or combinations thereof. The growth factor may comprise endothelial cell growth factor, epidermal growth factor, transforming growth factor-beta, insulin-like growth factor, platelet derived growth factor, periodontal ligament chemotactic factor, vascular endothelial growth factor, fibroblast growth factor, or combinations thereof. Suitable hormones include, but are not limited to human growth hormone, animal growth hormones, or combinations thereof. Suitable vitamins include, but are not limited to vitamin A, vitamin B6, vitamin B9, vitamin B12, vitamin C, vitamin D, vitamin K, or combinations thereof.

In some embodiments, the anti-inflammatory comprises a non-steroidal anti-inflammatory drug (NSAID), acetaminophen, an opioid, a cannabinoid, salicylic acid, lidocaine, or combinations thereof. Suitable NSAIDs include, but are not limited to ibuprofen, naproxen, diclofenac, celecoxib, mefenamic acid, etoricoxib, indomethacin, or combinations thereof. In some embodiments, the opioid is hydrocodone, oxycodone, morphine, oxymorphone, codeine, or combinations thereof. Suitable cannabinoids include, but are not limited to, THC, CBD, CBN. CBC, CBG, CBND, CBL, CBV, THCV, CBDV, CBCV, CBGV, CBGM, THCA, CBDA, CBNA, CBCA, CBGA, CBNDA, CBLA, CBVA, THCVA, CBDVA, CBCVA, CBGVA, or combinations thereof.

Suitable cannabinoids include, but are not limited to, tetrahydrocannabinol (THC), cannabidiol (CBD) and cannabinol (CBN). Other cannabinoids include for example, cannabichromene (CBC), cannabigerol (CBG) cannabinidiol (CBND), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM). As used herein THC, CBD, CBN, CBC, CBG, CBND, CBL, CBV, THCV, CBDV, CBCV, CBGV and CBGM refer to the decarboxylated form of the cannabinoid. Whereas THCA, CBDA, CBNA, CBCA, CBGA, CBNDA, CBLA, CBVA, THCVA, CBDVA, CBCVA, CBGVA and CBGAM (cannabigerolic acid monomethyl ether) refer to the corresponding acid form of the cannabinoid.

According to one or more embodiments, the plurality of particles is suspended in blood. The blood may include autologous blood, heparinized blood, heparinized blood with added calcium ions, whole blood, or whole blood with added calcium. In one or more embodiments, the concentration of the plurality of particles suspended in the blood is about 5 g/mL to about 750 g/mL, or any individual value or sub-range within this range.

Each of the plurality of particles may include absorbed blood. The absorbed blood may comprise autologous blood, heparinized blood, heparinized blood with added calcium ions, whole blood, or whole blood with added calcium.

In some embodiments, the implant is in the form of a dry flowable solid. The dry flowable solid may include particles having a particle size distribution of D₅₀ less than about 1 mm, or a mean size of about 0.005 mm to about 1.950 mm, or about 0.1 μm to about 2 mm, or about 1 μm to about 1 mm, or any individual value or sub-range within these ranges.

According to various embodiments, the plurality of particles is suspended in a liquid or gel medium. The liquid or gel medium may be water, a buffer (e.g., as described herein), saline, one or more hydrophilic polymers, or combinations thereof. In some embodiments, the plurality of particles suspended in the liquid medium is at a concentration of about 5 g/mL to about 750 g/mL, or any individual value or sub-range within this range.

In various embodiments, collagen implants have a collagen content of greater than 400 mg/g, about 400 mg/g to about 10,000 mg/g, or any individual value or sub-range within these ranges. Collagen implants according to embodiments herein may include DNA in an amount of less than 50,000 ng/g, about 5 ng/g to less than about 50,000 ng/g, or any individual value or sub-rang within these ranges. Collagen implants may include one or more GAGs in an amount of greater than 100 μg/g, greater than about 100 μg/g to about 8,000 μg/g, or any individual value or sub-range within these ranges. Collagen implants may include a phospholipid having a phospholipid count of less than 3,000 μM/g, about 1 μM/g to less than about 3,000 μM/g, or any individual value or sub-range within these ranges. In some embodiments, collagen implants include pepsin in an amount of less than 12.5 mg/g, about 0.1 mg/g to less than about 12.5 mg/g, or any individual value or sub-range within these ranges. In some embodiments, the collagen implants comprises a collagen content of greater than 400 mg/g, a GAG content of greater than 100 μg/g, a DNA content of less than 50,000 ng/g, a phospholipid count of less than 3,000 μM/g and a pepsin content of less than 12.5 mg/g.

The collagen implant includes a structural integrity sufficient to receive therethrough one or more sutures for securement to the ruptured or damaged tissue. The collagen implant is configured to interact with the subject's body to develop a network of capillaries, arteries, and veins at the tissue tear or rupture. Well-vascularized connective tissues heal as a result of migration of fibroblasts into the collagen implant. The methods and systems of the present disclosure establish a link between the damaged tissue, either by encircling the torn tissue or connecting with it, fostering the mending process of the ruptured or torn tissue while preserving its integrity and structure. The collagen implant may function either as an insoluble or biodegradable regulator of cell function or as a delivery vehicle of a supporting structure for cell migration or synthesis, and may provide a network or structure to facilitate cell in growth and vascularization.

The collagen implant may be configured to provide a connection between the ruptured or torn portions of the tissue and fibers, or form around the torn tissue, after injury, and encourages the migration of appropriate healing cells to form scar and new tissue. The collagen implant may therefore be a substitute for a clot. The collagen implant is thus implanted adjacent to ruptured or torn portions of tissues, between the ruptured or torn portions of the tissue, or wrapped around the tissue. In other embodiments, the collagen implant, in combination with the suture assemblies, is capable of forming a connection between the implant and bone, between soft tissue and bone, and/or being formed around the torn tissue such that the integrity and structure of the tissue is maintained. The collagen implant is therefore designed to stimulate cell proliferation and extracellular matrix production in the space near or adjacent ruptured tissue and the gap between the ruptured ends of the tissue or the tear in the tissue site, thus facilitating healing and regeneration.

### Methods of Preparing Injectable Collagen Implants

Further described herein according to various embodiments are methods of preparing implants configured for injection into a repair site in or near a target tissue. The methods may include preparing a collagen slurry according to one or more embodiments described herein. The methods may further include heating the collagen slurry to form a self-assembled collagen slurry. In some embodiments, heating the collagen slurry includes heating to an elevated temperature to cause self-assembly of the collagen molecules within the slurry. Heating the collagen slurry may be at a temperature of about 27° C. to about 50° C., about 30° C. to about 40 C, about 30° C. to about 37° C., or any individual value or sub-range within these ranges. The heating may occur for up to 24 hours, up to 12 hours, up to about 8 hours, about 1 hour to about 24 hours, or any individual value or sub-range within these ranges.

In some embodiments, the self-assembled collagen slurry is freeze-dried to produce the collagen implant. Before the freeze-drying process proceeds, the self-assembled collagen slurry may be cooled to a temperature of about 4° C. or less in a refrigerator or freezer, with or without the aid of dry ice and/or liquid nitrogen. Suitable freeze-drying methods include lyophilization as described herein above. Freeze-drying the self-assembled collagen slurry is suitable to achieve a residual moisture level of less than about 5%, or about 1% to about 5% of the total weight of the self-assembled collagen slurry.

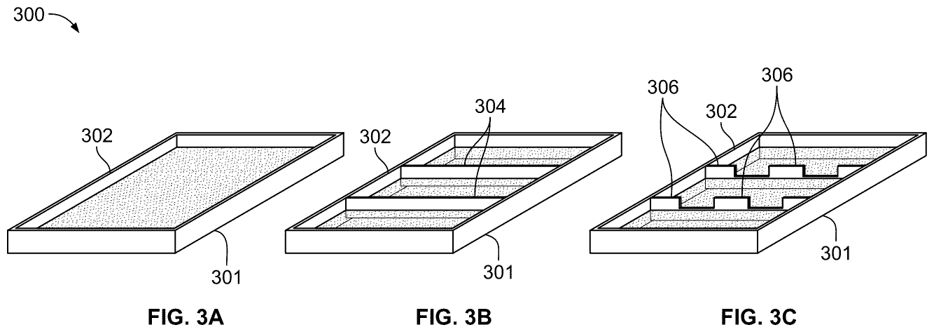

The collagen slurry may be introduced into a tray 300 prior to heating the collagen slurry, or prior to freeze-drying the collagen slurry. Embodiments of a tray 300 used for heating, self-assembly, cooling, and/or freeze-drying is shown in FIGS. 3A-3C. In some embodiments, the tray includes a floor 301, and an outer frame 302 disposed around a periphery of the floor. In some embodiments, for example, as shown in FIGS. 3B and 3C, tray 300 includes an inner grid 304, 306 received within the outer frame on top of the floor. The inner grid may be a single component having fixed, defined compartments, or a plurality of components movable to form various compartments within the tray. In some embodiments, the inner grid does not extend all the way through the self-assembled collagen slurry or the collagen implant. The inner grid may divide the interior of tray 300 into equal portions, for example, as shown in FIG. 3B, and/or the inner grid may be formed of pieces that do not extend the entire depth of tray 300 interior, for example, as shown in FIG. 3C. The inner grid may extend partially, or about 20% to about 60%, about 30% to about 50%, or any individual value or sub-range within these ranges through the self-assembled collagen slurry or the collagen implant.

In some embodiments, the self-assembled collagen slurry is moved while in the tray from the heating operation to the refrigerator, freezer, and/or freeze dryer chamber. The resulting collagen implant may be in the form of a sheet (e.g., FIG. 3A) having a thickness of about 0.1 cm to about 10 cm, about 0.5 cm to about 8 cm, about 1 cm to about 4 cm, or any individual value or sub-range within these ranges. In embodiments, the resulting collagen implant may be in the form of a plurality of sheets or cuboids (e.g., FIG. 3B), or may be divided as shown in FIG. 3C.

Methods of preparing implants suitable for injection may further include dividing the sheet into a plurality of particles. Dividing the sheet into the plurality of particles may include cutting the sheet with a blade, bandsawing, die cutting, breaking, grating, shredding, grinding, milling, or blending the collagen implant, or combinations thereof. Each of the plurality of particles prepared by the methods described herein may have a mean size of less than about 10 mm, less than about 5 mm, less than about 2 mm, less than about 1 mm, less than about 1 μm, less than about 0.1 μm, about 0.1 μm to about 10 mm, or any individual value or sub-range within these ranges.

According to various embodiments, the collagen implant is in the form of a plurality of particles as described herein. At least two particles of the plurality of particles are a different shape and/or size. The collagen implant may be in the form of a granules, extrudates, powders, pellets, multi-particulates (e.g., coated sub-units, bi-layer sub-units, multi-layer sub-units), minitablets, microcapsules, cuboids, hexoids, rhomboids, spheres, microspheres, cylinders, ovals, fibers, and/or combinations thereof. The methods of preparing the collagen implants may further include sieving the plurality of particles to a mean size of less than about 10 mm, less than about 5 mm, less than about 2 mm, less than about 1 mm, less than about 1 μm, less than about 0.1 μm, about 0.1 μm to about 10 mm, or any individual value or sub-range within these ranges. Sieving may be by dry sieving, vibratory sieving, centrifugal sieving, air jet sieving, tapping sieving, rotary sieving, multilayer sieving, or combinations thereof.

Collagen implants prepared by these methods may have a porosity of at least about 50%, at least about 60%, at least about 70%, at least about 80%, about 50% to 99%, about 80% to 99%, or any individual value or sub-range within these ranges. In some embodiments, each of the plurality of particles comprises a porosity of at least about 50%, at least about 60%, at least about 70%, at least about 80%, about 50% to 99%, about 80% to 99%, or any individual value or sub-range within these ranges. In some embodiments, each of the plurality of particles has a porosity of less than about 10%, about 1% to about 10%, or any individual value or sub-range within these ranges.

According to various embodiments, methods of preparing implants configured for injection are in the form of a dry flowable solid. The dry flowable solid may include particles having a particle size distribution of D₅₀ less than about 1 mm, or a mean size of about 0.005 mm to about 1.950 mm, or about 0.1 μm to about 2 mm, or about 1 μm to about 1 mm, or any individual value or sub-range within these ranges.

Methods of preparing implants as described herein may further include suspending the plurality of particles in a liquid or gel medium. The liquid or gel medium may include water, a buffer, saline, one or more hydrophilic polymers, or combinations thereof. The plurality of particles in the liquid medium may be at a concentration of about 5 g/mL to about 750 g/mL, or any individual value or sub-range within this range.

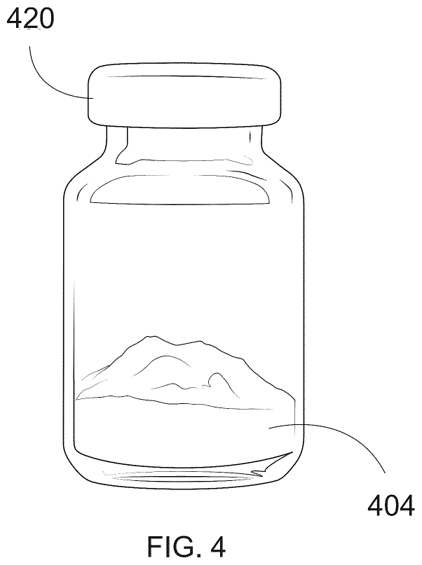

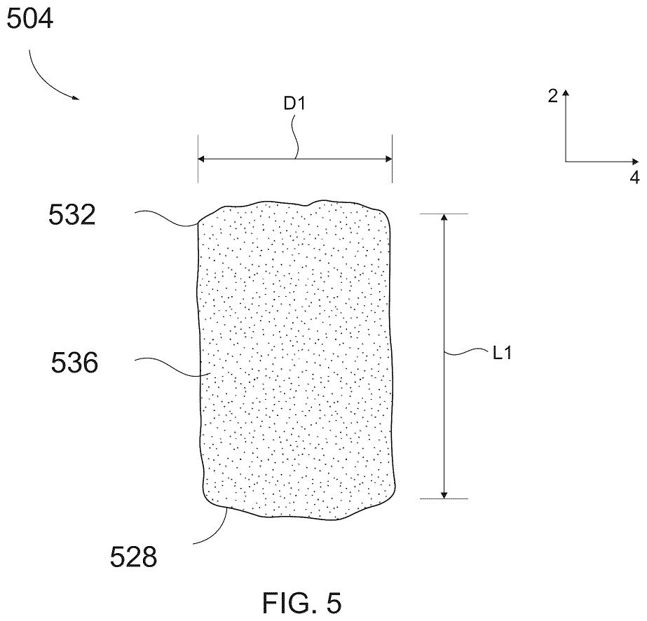

Collagen implants according to embodiments herein may have a size and shape chosen to complement the specific anatomical tissue present. FIGS. 4 and 5 depict the implant 404, 504 according to embodiments of the present disclosure. Referring to FIGS. 4 and 5, the implant 404 is a powder (or plurality of collagen particles) configured to be inserted or injected into a repair site. In some embodiments, the collagen implant is injected at the repair site and then self assembles into a solid or semi-solid structure that conforms to the shape of the tissue (e.g., to fill the tear or rupture, to simulate a clot, etc.). For example, the collagen implant may be particles in the form of a powder, flakes, and/or chunks, configured for insertion or injection at the repair site. In one or more embodiments, the particles may have a mean particle size D₅₀ of less than about 70 mm, or about 0.005 μm to about 70 mm, about 1.0 μm to about 50 mm, about 5.0 mm to about 25 mm, about 500 μm to about 2,000 μm, or any individual value or sub-range within these ranges.

In at least one embodiment, the collagen implant has a particle size suitable to be expelled through a standard male luer-lock opening, the opening having an inner diameter of approximately 2.0 mm (per ISO594-2(1998) or ISO 80369-1(2018)). In one or more embodiments, the implant may be formed of particles, also referred to herein as a dry flowable solid, having a particle size distribution D₅₀ of less than about 2 mm, or a mean size of about 0.005 μm to about 1.950 mm, or about 0.1 μm to less than about 2 mm, or about 1 μm to about 1 mm, or any individual value or sub-range within these ranges. In various embodiments, the collagen implant 104 (or each of the plurality of particles) may have a pore size of greater than about 10 μm, greater than about 10 μm to about 1,000 μm, or an individual value or sub-range within these ranges.

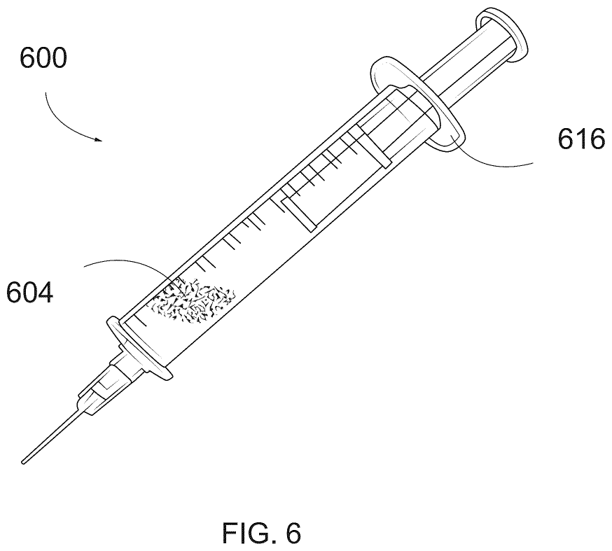

In the illustrated embodiment of FIG. 4, for surgical application, the implant 404 is provided in a separate container 420, such as a bottle, pouch, tube, or other known containers. The implant 404 may then be collected and loaded onto or in the delivery device (as shown in FIGS. 6-10F) to be inserted into the repair site. In another embodiment, however, the implant 404 may be preloaded into the delivery device. For example, the delivery device may be a single or double-chamber syringe. The implant 404 is hydrophilic and capable of absorbing plasma, blood, other body fluids, liquid, hydrogel, or other material the scaffold either comes into contact with or is added to the scaffold.

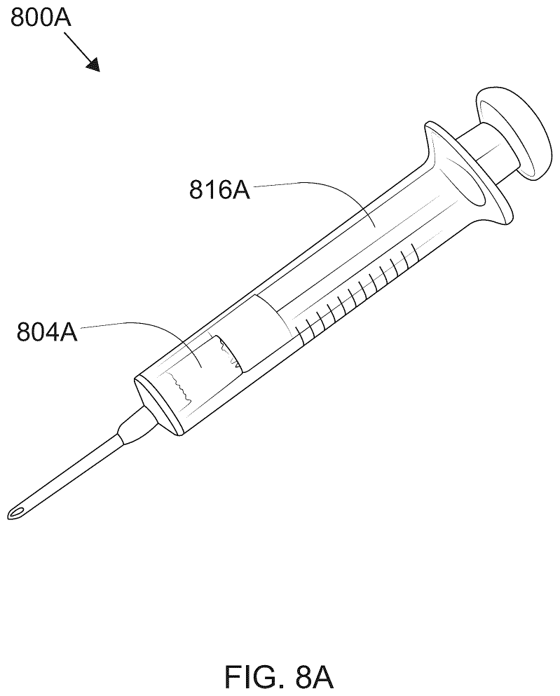

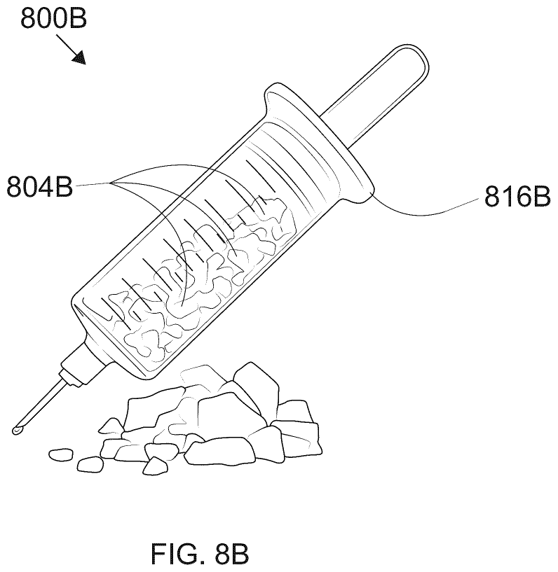

FIG. 5 depicts the implant 504 according to another embodiment of the present disclosure. Referring to FIG. 5, the implant 504 may be one or more pieces having a lower surface 528, an upper surface 532 opposite the lower surface 528, and a side wall 536 that extends from the lower surface 528 to the upper surface 532. In this configuration, the implant 504 may therefore be provided as a single piece (as shown in FIG. 8A) providing enough material to facilitate repair, or as multiple pieces (as shown in FIG. 8B), allowing the ability to choose the desired amount of pieces based on the size of the defect.

The implant 504 may have a length L1 along a longitudinal axis 2, which extends from the lower surface 528 to the upper surface 532. The length L1 may range from 5.0 mm to 70.0 mm. The implant 504 may have a cross-sectional dimension D1, which extends along a latitudinal axis 4 and is perpendicular to the length L1, that ranges from 5.0 mm to 25.0 mm. The implant 504 has a total volume that ranges from 98.17 mm³ to 34,361.17 mm³.

In the illustrated embodiment of FIG. 5, the implant 504 may be provided in different geometrical shapes to enable various configurations of the surgical procedure. For example, in one embodiment, the implant 504 may be in the form of one or more sheets, wherein each sheet has a thickness of less than half of the length of the next shortest edge. In another embodiment, the implant 504 may be in the form of one or more cuboids, wherein each cuboid has a thickness of greater than half the length of the next shortest edge. In other embodiments, the implant 504 may be in the form of one or more cylinders, cones, spheres (full or partial), and may be tailored, straight, concave, or convex. In another embodiment, the implant 504 may include a plurality of pieces (i.e. a plurality of implants), wherein each piece is a different shape and/or size as outlined above.

The implant 504 may have a denaturation temperature between 35° C. and 43° C. Further, the implant 504 may have a porosity that ranges from about 50% to 97% porosity. However, in certain instances, a lower porosity may be used between, for example, 20% and 50% porosity. Each pore has a diameter of at least about 10 μm, at least about 20 μm, greater than about 10 μm to about 1,000 μm, or any individual value or sub-range within these ranges, in order to facilitate cell migration. In one embodiment, the pores are uniform in size along the body of the implant 504. In another embodiment, the pores may form a gradient in size along the body of the implant 504. For example, the gradient may increase or decrease from one edge to the opposite edge. In another example, the gradient may increase or decrease towards the center such that the largest or smallest pores are in the center of the implant 504. In another example, the pores may be larger or smaller on one surface than another. In yet another embodiment, the core of the implant 504 may have smaller or larger pores than the outer surfaces of the implant 504.

In one embodiment, for surgical application, the implant 504 is provided in a separate container, such as a bottle, pouch, tube, or other known containers. The implant 504 may then be collected and loaded onto or in the delivery device (as shown in FIGS. 8A-10F) to be inserted into the repair site.

The implant 504 is hydrophilic and capable of absorbing plasma, blood, other body fluids, liquid, hydrogel, or other material the implant 504 either comes into contact with or is added to the implant 504. In the illustrated embodiment, the implant 504 is treated with a repair material prior to insertion into a repair site. As illustrated, the repair material is blood. Specifically, the implant 504 is treated with 5-10 mL of autologous blood. The implant 504 may be either soaked in the repair material or the repair material is injected into the implant 504 prior to or during implantation into the repair site. In other examples, the repair material may be an autologous or allogeneic blood composition, plasma or other fluids either present within the repair site, added to the implant 504 either before or after the implant 504 is inserted into the repair site, or added into the repair site

### Tissue Repair Systems, Delivery Devices, and Kits

Further described herein are tissue repair systems, delivery devices, and kits. A tissue repair system according to embodiments herein may include a collagen implant as described herein configured for injection into a target tissue, and a delivery device configured to deliver the collagen implant to the target tissue. The target tissue may include cartilage, for example a meniscus or rotator cuff. The delivery device may include an elongated body having a proximal end, a distal end, a channel that extends from the proximal end to the distal end, and an actuator in the channel, wherein the actuator is configured to cause the collagen implant to exit the distal end of the body. In some embodiments, the delivery device is a syringe having a single chamber. In some embodiments, the delivery device is a syringe having a double chamber.

Repair systems may include a collagen implant and one or more suture assemblies configured to secure the collagen implant in a repair site at a tissue of interest. The collagen implant may be a compressible and biocompatible implant that is configured to absorb fluid, such as blood and/or blood components. The collagen implant may be configured to heal or repair tissue. In some embodiments, the collagen implant may comprise a three-dimensional (3-D) scaffold for repairing the ruptured or torn tissue. Tissue here may include ligaments, cartilage, and tendons, such as an ACL, meniscus or rotator cuff, etc.

The collagen implant may be preloaded into the delivery device. In at least one embodiment, the collagen implant is stored in a container separate from the delivery device. The elongated body may have a port for receiving fluid such as an injectable collagen implant or slurry.

Another embodiment of a delivery device for tissue repair may include an elongated body having a proximal end, a distal end, a channel that extends from the proximal end to the distal end, and an actuator in the channel. The delivery device may house a collagen implant configured for injection into a target tissue according to various embodiments described herein. In one or more embodiments, the delivery device includes an actuator configured to cause the collagen implant to exit the distal end of the body. In this embodiment, the elongated body may have a port for receiving fluid. The delivery device may be a syringe having a single chamber or a syringe having a double chamber.

According to further embodiments herein is a tissue repair kit, including a tissue repair system as described above, a collagen implant configured for injection into a target tissue in accordance with various embodiments described herein, and instructions for use. The delivery device may include an elongated body having a proximal end, a distal end, a channel that extends from the proximal end to the distal end, and an actuator in the channel, wherein the actuator is configured to cause the collagen implant to exit the distal end of the body. In some embodiments, the delivery device is a syringe having a single chamber or a double chamber. In one or more embodiments, the elongated body has a port for receiving fluid. The collagen implant may be preloaded in the delivery device and/or stored in a container separate from the delivery device. In at least one embodiment, the collagen implant is configured to be combined with blood prior to delivery into the target tissue. In one or more embodiments, the collage implant is configured to be combined with blood in the delivery device prior to delivery into the target tissue.

According to various embodiments, systems and kits as described herein may further include at least one suture assembly configured to be inserted at a repair site in order to repair the ruptured or damaged tissue. The collagen implant may have a structural integrity sufficient to receive therethrough one or more sutures for securement to the ruptured or damaged tissue. The implant allows the subject's body to develop a network of capillaries, arteries, and veins at the tissue tear or rupture. Well-vascularized connective tissues heal as a result of migration of fibroblasts into the implant. The methods and systems of the present disclosure establish a link between the damaged tissue, either by encircling the torn tissue or connecting with it, fostering the mending process of the ruptured or torn tissue while preserving its integrity and structure. The implant may function either as an insoluble or biodegradable regulator of cell function or as a delivery vehicle of a supporting structure for cell migration or synthesis, and may provide a network or structure to facilitate cell in growth and vascularization.

In some embodiments, the implant includes a three-dimensional (3-D) scaffold for repairing a ruptured or torn tissue. The implant may be combined to provide a connection between the ruptured or torn portions of the tissue and fibers, or form around the torn tissue, after injury, and encourages the migration of appropriate healing cells to form scar and new tissue. The implant is a bioengineered substitute for a clot. The implant is thus implanted between the ruptured or torn portions of the tissue, or wrapped around the tissue, or placed adjacent to the ruptured or torn tissue. In other embodiments, the implant is capable of forming a connection between bone, or forming around the torn tissue such that the integrity and structure of the tissue is maintained. This implant is therefore designed to stimulate cell proliferation and extracellular matrix production in the gap between the ruptured ends of the tissue or the tear in the tissue, thus facilitating healing and regeneration.

FIG. 6 depicts a tissue repair system 600 utilized in tissue repair. The repair system 600 includes a collagen implant 404, 604, which is configured to be inserted at a repair site in order to repair the ruptured or damaged tissue. The system 600 further includes a delivery device 616 configured to carry the collagen implant 404, 604 for delivery at the repair site.

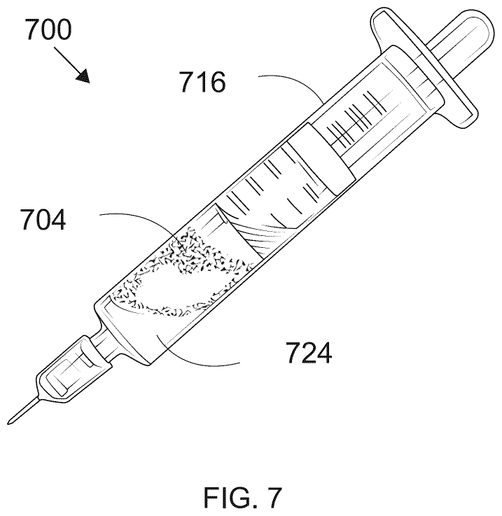

FIG. 7 is a diagrammatic representation of a tissue repair system 700 utilizing the implant 404 shown in FIG. 4. In this embodiment, however, the implant 404, 704 may be preloaded into the delivery device 716. In this configuration, the delivery device 716 may be a single or double-chamber syringe. The implant 404, 704 may be treated with a repair material prior to insertion into a repair site. As illustrated, the repair material 724 is blood. Specifically, the implant 404, 704 is treated with 5-10 mL of autologous blood. The implant 404, 704 may be either soaked in the repair material 724 or the repair material 724 is injected into the implant 404, 704 prior to or during implantation into the repair site. In other examples, the repair material 724 may be an autologous or allogeneic blood composition, plasma or other fluids either present within the repair site, added to the implant 404, 704 either before or after the implant 404, 704 is inserted into the repair site, or added into the repair site.

FIGS. 8A and 8B depict a tissue repair system for the implant 504, 804A, 804B according to other embodiments of the present disclosure. The implant 504, 804A, 804B may be preloaded into the delivery device 816A, 816B. The delivery device 816A, 816B may be an open-bore syringe. In other embodiments, the delivery device may be any surgical delivery device known in the art including paste tubes, hose or piping-like tubes, and squeeze bottles.

Referring to FIGS. 8A and 8B, the implant 504, 804A, 804B may be provided as a single piece (as shown in FIG. 8A) providing enough material to facilitate repair, or as multiple pieces (as shown in FIG. 8B), allowing the ability to choose the desired amount of pieces based on the size of the defect.

In one embodiment, for surgical application, the implant 504, 804A, 804B is provided in a separate container, such as a bottle, pouch, tube, or other known containers. The implant 504, 804A, 804B may then be collected and loaded onto or in the delivery device 816A, 816B to be inserted into the repair site. In another embodiment, the implant 504, 804A, 804B may be preloaded into the delivery device 816A, 816B. In the illustrated embodiment, the delivery device 816A, 816B is an open-bore syringe. However, in other embodiments, the delivery device may be any surgical delivery device known in the art including paste tubes, hose or piping-like tubes, and squeeze bottles.

The implant 504, 804A, 804B is hydrophilic and capable of absorbing plasma, blood, other body fluids, liquid, hydrogel, or other material the implant 504, 804A, 804B either comes into contact with or is added to the implant 504, 804A, 804B. In the illustrated embodiment, the implant 504, 804A, 804B is treated with a repair material prior to insertion into a repair site. As illustrated, the repair material is blood. Specifically, the implant 504, 804A, 804B is treated with 5-10 mL of autologous blood. The implant 504, 804A, 804B may be either soaked in the repair material or the repair material is injected into the implant 504, 804A, 804B prior to or during implantation into the repair site. In other examples, the repair material may be an autologous or allogeneic blood composition, plasma or other fluids either present within the repair site, added to the implant 504, 804A, 804B either before or after the implant 504, 804A, 804B is inserted into the repair site, or added into the repair site.

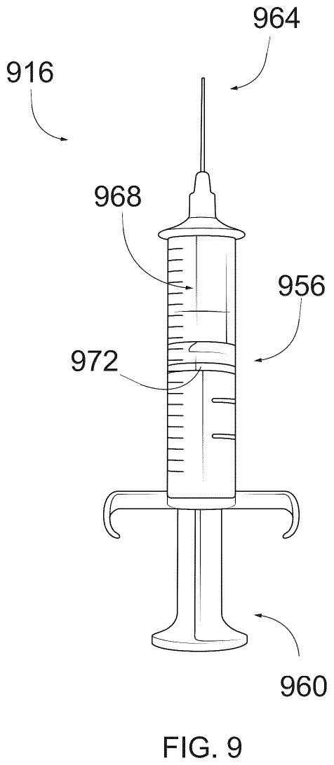

Referring to FIG. 9, a delivery device 916 is configured to carry the collagen implant 404 for delivery to the site of the torn or ruptured tissue. In this instance, the collagen implant is a plurality of collagen particles (e.g., each formed of a self-assembly of collagen fibers, or formed by grinding, cutting, dividing, etc.). The delivery device 916 has an elongated body 956, a proximal end 960, a distal end 964 opposite the proximal end 960, and a channel 968 that extends from the distal end 964 toward the proximal end 960. The channel 968 defines a cross-sectional dimension that is shaped and sized to carry the collagen implant 404. The delivery device 916 includes a moveable rod 972 in the channel 968 and configured to eject the collagen implant 404 from the distal end 964 of the delivery device 916 into the tissue site.

In the illustrated embodiment, the delivery device 916 is a syringe having a single chamber. In alternative embodiments, the delivery device 916 is a double-chamber syringe. The syringe 916 may hold both the implant 404 and the implant 404 mixed with autologous blood in place within the elongated body 956 of the syringe. The syringe may include a plunger configured to push the implant 404 into a repair site such that the implant 404 is positioned adjacent to at least one torn or ruptured end of the tissue. In alternative embodiments, the delivery device 916 may include a cannula, such as an arthroscopic cannula, a container, and a pressure pump. In yet another embodiment, the implant 404 may be inserted into the repair site without the use of arthroscopic equipment and instead through an open surgical procedure.

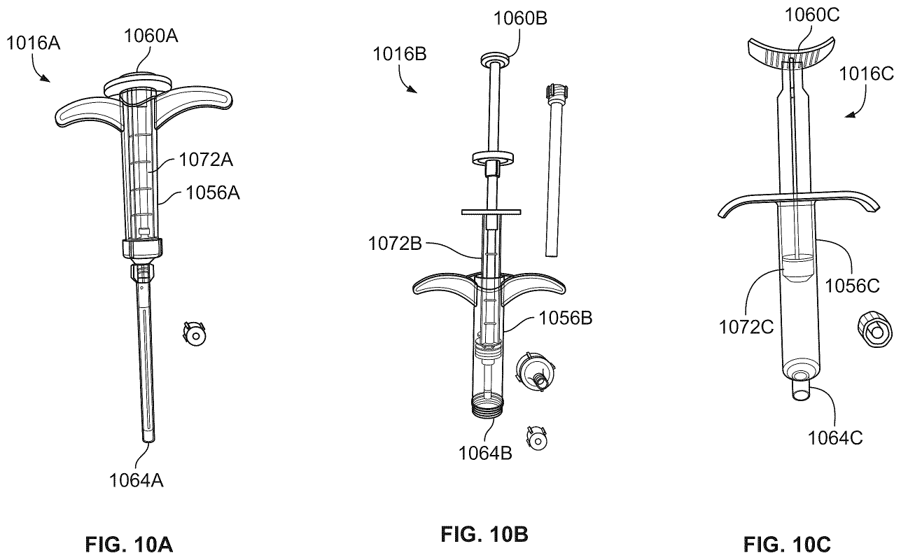

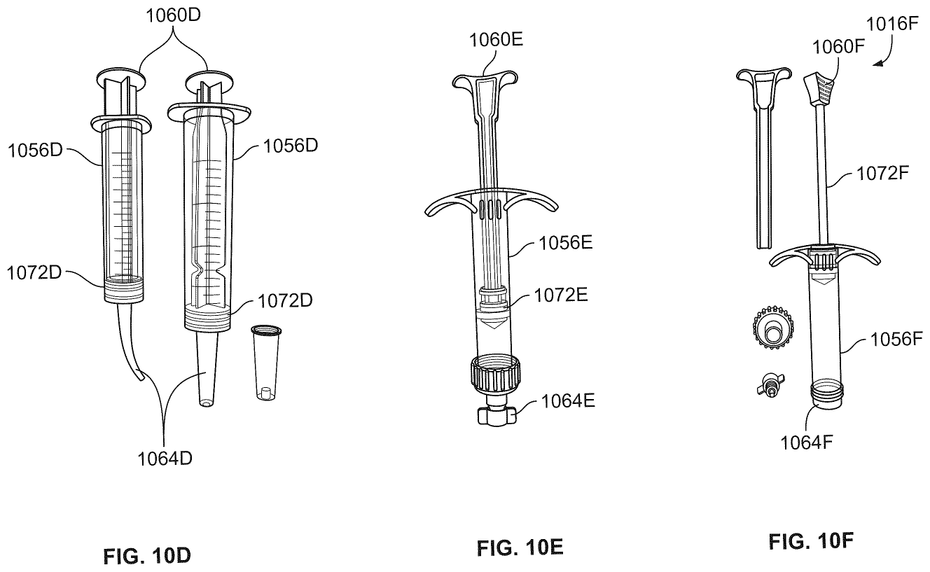

Embodiments of other delivery devices 1016A-F configured to carry the collagen implant 404, 504 for delivery to the repair site of the torn or ruptured tissue are shown in FIGS. 10A-10F. The delivery devices 10A-10F are suitable for injecting implant particles 404 (e.g., mixed with autologous blood) and for injecting an implant 504 sheet (e.g., a compressible sponge, large pieces of implant material, etc.) into the repair site. Each delivery device 1016A-F has an elongated body 1056A-F, a proximal end 1060A-F, a distal end 1064A-F opposite the proximal end 1060A-F, and an internal channel that extends through the elongated body 1056A-F from the distal end 1064A-F toward the proximal end 1060A-F. The channel defines a cross-sectional dimension that is shaped and sized to carry the collagen implant 404, 504. The delivery device 1016A-F includes a moveable rod 1072A-F in the channel and configured to eject the collagen implant 404, 504 from the distal end 1064A-F of the delivery device 1016A-F into the tissue site.

In the illustrated embodiments, each delivery device 1016A-F is a syringe having a single chamber. In alternative embodiments, each delivery device 1016A-F may be a double-chamber syringe. The syringe 1016A-F may hold both the implant 404, 504 and the implant 404, 504 mixed with autologous blood in place within the elongated body 1056A-F of the syringe. The syringe may include a plunger configured to push the implant 404, 504 into a repair site such that the implant 404, 504 is positioned adjacent to at least one torn or ruptured end of the tissue. In alternative embodiments, the delivery device 1016A-F may include a cannula, such as an arthroscopic cannula, a container, and a pressure pump. In yet another embodiment, the implant 404, 504 may be inserted into the repair site without the use of arthroscopic equipment and instead through an open surgical procedure.

### Methods for Repairing Tissue Using Injectable Collagen Implants

Further described herein are methods for repairing tissue using implants configured for injection. It should be noted that the methods disclosed herein may be used for multiple tissue types, including various ligaments and tendons. Collagen slurries as described herein may be implanted into a subject of the same or a different species. The terms “xenogeneic” and “xenograft” refer to cells or tissue which originates with or is derived from a species other than that of the recipient.

In one or more embodiments, methods for repairing a tissue include forming a portal in a repair site of the tissue. The methods may further include injecting or inserting a delivery device comprising a collagen implant composition according to embodiments herein through the portal such that a distal end of the delivery device is positioned close to the tissue.

In some embodiments, to secure the collagen and/or collagen implant composition as described herein to the rupture or tear, collagen and/or collagen implant composition is mixed with blood, platelet rich plasma, white blood cells, etc. to form a viscous paste or putty mixture, depending on the ratio. Suitable volumetric ratios include, but are not limited to about 1:1 to about 100:1 of the volume of the collagen or collagen implant composition to the volume of the blood, platelet rich plasma, white blood cells, etc., or any individual value or sub-range within this range. When collagen or a collagen implant composition in an amount of about 3 ml to about 1,000 ml, or any individual value or sub-range therein, is mixed with a volume of about 3 ml to about 10 ml of blood, platelet rich plasma, white blood cells, etc., the resulting mixture may be viscous enough to be expelled from the delivery device, but stable enough to stay within the confines of the anatomical fields into which it is injected. Suitable viscosities for the collagen, collagen composition, or for the mixture with blood, platelet rich plasma, white blood cells, etc., include, but are not limited to, about 1 cP to about 10 cP, about 1.2 cP to about 8 cP, about 3.5 cP to about 5.5 cP, or any individual value or sub-range within these ranges. Over time, the intended formation of a blood clot, for example, by coagulation, may further stabilize the implant and prevent it from being ‘washed away’. In some embodiments, injectable implant compositions as described herein include calcium salts(s) in an amount of about 0.1% by weight to about 25% by weight, about 0.8% by weight and 4% by weight, or any individual value or sub-range within these ranges, to increase the speed of the blood clot formation and to help with securing the implant in place.

In at least one embodiment, the methods of repairing a tissue include manipulating the delivery device to release the collagen implant from the distal end of the delivery device and to the tissue such that the collagen implant rests within or near the tissue. Suitable tissues for repair by collagen implants as described herein include, but are not limited to, a rotator cuff, an anterior cruciate ligament, or a meniscus.

In some embodiments, the collagen implant is combined with blood prior to inserting the delivery device through the portal. In at least one embodiment, the methods include adding the collagen implant to the delivery device, and adding blood to the delivery device prior to inserting the delivery device through the portal.

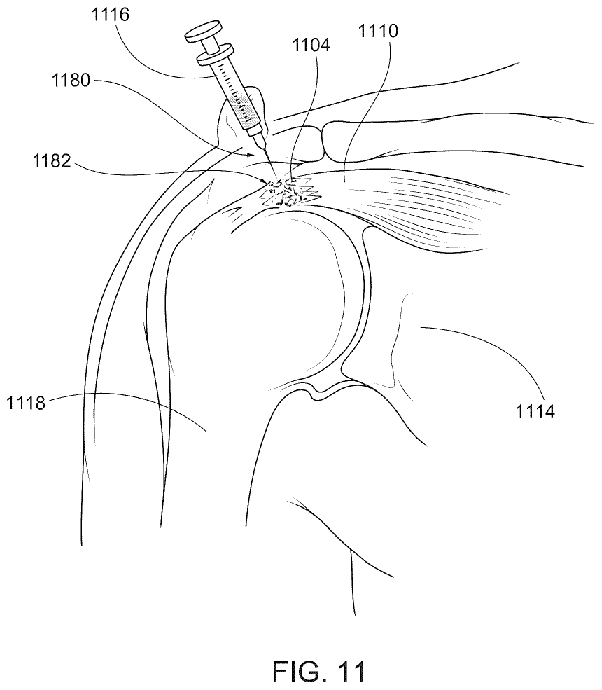

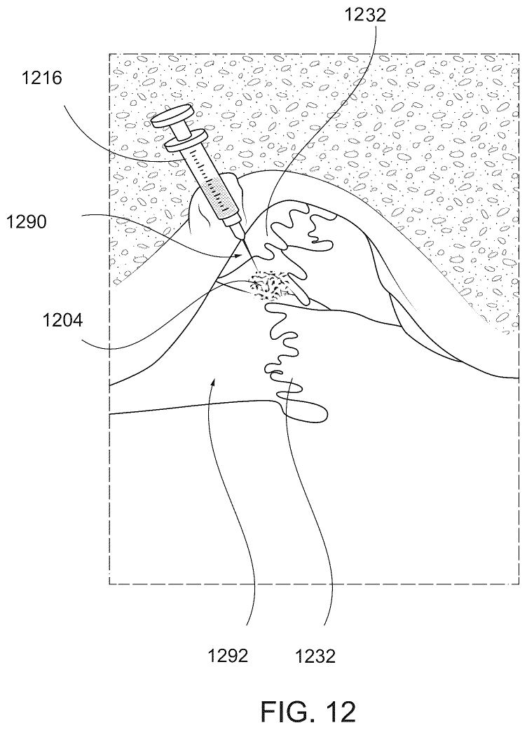

Referring to FIGS. 11 and 12, aspects of the invention relate to methods for repairing a ruptured or torn tissue utilizing the embodiment shown in FIGS. 4 and 5. In some embodiments are methods for repairing a rotator cuff tendon. Such methods include attaching a first fixation device to a humerus, and attaching a second fixation device to the rotator cuff tendon. A flexible construct may be connected to the two fixation devices. In embodiments, the methods further include injecting a collagen implant according to embodiments herein on or near the rotator cuff such that the collagen implant rests between torn ends of the rotator cuff tendon. In some embodiments, the first fixation device is attached only indirectly to the collagen implant. The collagen implant may be positioned between a ruptured portion of the rotator cuff via the flexible constructs. The collagen implant may be combined with blood prior to injecting the collagen implant.

FIG. 11 shows an injury to a ruptured or turn rotator cuff. A portal 1180 in the joint is formed to access a rotator cuff at a repair site 1182. In the illustrated embodiment, the implant 404, 504, 1104 is contained in the delivery device 1116. The delivery device 1116 is inserted into the portal so that a distal end of the delivery device 1116 is positioned close to the rotator cuff. The collagen implant 404, 504, 1104 is manipulated such that the implant 404, 504, 1104 exits the distal end of the delivery device 1116 in order to position it proximate the rupture or tear of the rotator cuff. The sutures are then inserted into the tendon and through the collagen implant 404, 504, 1104 to secure the rupture or tear of the rotator cuff. The collagen implant 404, 504, 1104 may be a semi-slide when injected, or may be in the form of particles that assemble and form a solid structure following injection. The sutures maintain the tendon in a desired position and are suitable to maintain the implant 404, 504, 1104 Repair material 1124 is then injected into the delivery device with the implant 404, 504, 1104 in position prior to, during, or following injection of the implant 404, 504, 1104 into the repair site 1182 utilizing the same delivery device 1116. In another embodiment, the implant 404, 504, 1104 is pretreated with a repair material to form a mixture prior to insertion into the repair site 1182.

According to further embodiments, described herein are methods for repairing an anterior cruciate ligament (ACL). Such methods may include drilling a tibial tunnel in a tibia. The methods may further include drilling a femoral tunnel in a femur so that the femoral tunnel and tibia tunnels are aligned. The methods may further include placing a bone fixation device on a femoral cortex, wherein a first and second set of sutures are attached to the bone fixation device and pass through the femoral tunnel. The methods for repairing an ACL may further include securing the first set of sutures onto the ACL with a rupture or tear.

The method of repairing the ACL may further include injecting a collagen implant according to embodiments herein at or near the second set of sutures. In embodiments, the method includes passing the first and second set of sutures, attached to a second bone fixation device, through the tibial tunnel. The bone fixation device may be secured in place on the tibial cortex. In some embodiments, methods of ACL repair may comprise observing growth of an ACL tissue and meniscus at or near a rupture or tear of the ACL and the meniscus. In some embodiments, the collagen implant is combined with blood prior to injecting the collagen implant.

Methods of ACL repair may further include coupling the collagen implant to the femur with the bone fixation device. In embodiments, the methods comprise positioning the collagen implant between a ruptured end of the ligament and the femur and attaching the first fixation device to the femur and the second bone fixation device to the tibia. Positioning the collagen implant may be between a ruptured portion of the ligament. In some embodiments, the methods include attaching a bone fixation device (e.g., an extracortical bone fixation device) indirectly to the collagen implant.

Another embodiment for repairing one or one tears or ruptures in an ACL is described herein. The methods may include coupling a first suture assembly and a second suture assembly to a bone fixation device. A first portion of the first set of sutures may be secured to a ruptured or torn ACL. In embodiments, the methods include positioning the bone fixation device on a surface of the femur so that the first and second sets of sutures extend through a femoral tunnel.

A collagen implant according to embodiments herein may be injected proximate the ACL and a lateral or medial meniscus. The collagen implant may be combined with blood prior to injecting the collagen implant.

The collagen implant may be secured with a second bone fixation device with the second set of sutures to a surface of the tibia so that the second set of sutures extends through a tibial tunnel. In some embodiments, methods include observing growth of an ACL tissue and meniscus at or near a rupture or tear of the ACL and the meniscus. Observing growth may include obtaining and analyzing an MRI image of the ACL tissue and meniscus at the tissue site.

Embodiments may further comprise coupling the collagen implant to the tibia with a second bone fixation device. The collagen implant may be positioned between a ruptured end of a ligament and a femur and attaching the first fixation device to the femur and the second bone fixation device to the tibia. In some embodiments, the first fixation device is attached only indirectly to the collagen implant.

Another embodiment of repairing an ACL comprises securing a first portion of a first suture assembly to a ruptured or torn ACL. A second portion of the first suture assembly may be secured relative to a femur such that the first set of sutures extends from the ruptured or torn ACL and through a femoral tunnel to a bone fixation device. A first portion of a second set of sutures may be secured relative to a tibia so that a suture tail of the second set of sutures extends through a tibial tunnel into a region near the anterior cruciate ligament. The methods may include securing a second portion of the second set of sutures relative to a femur at the bone fixation device so that the second set of sutures extends into a femoral tunnel alongside or adjacent the ACL.

Methods may further comprise coupling the collagen implant to the tibia with a second fixation device. The collagen implant may be positioned between a ruptured end of a ligament and a femur and attaching the first fixation device to the femur and the second fixation device to the tibia. The first fixation device may be attached only indirectly to the collagen implant.

In embodiments, the methods further include injecting a collagen implant according to embodiments herein at a location adjacent the rupture or torn portion of the ACL. Blood from the collagen implant is permitted to emanate from the collagen implant into the region near the anterior cruciate ligament and a tear or rupture in either or both of a lateral meniscus and a medial meniscus.

In some embodiments, the methods include observing healing of the rupture or tear of ACL and the lateral meniscus or medial meniscus. Observing growth of the ACL tissue and meniscus tissue may be at or near a rupture or tear of the ACL and the meniscus.

Referring to FIG. 12, the injury is a ruptured or ACL. A portal 1290 is formed to access the ACL at a repair site 1292. In the illustrated embodiment, the implant 404, 504, 1204 is contained in the delivery device 1216. The delivery device 116 is inserted into the portal so that a distal end of the delivery device 1216 is positioned close to the ACL. The collagen implant 404, 504, 1204 is manipulated such that the implant 404, 504, 1204 exits the distal end of the delivery device 1216 in order to position it proximate the rupture or tear of the ACL. The collagen implant 404, 504, 1204 is then secured at the rupture or tear of the ACL. The collagen implant 404, 504, 1204 may be a semi-slide when injected, or may be in the form of particles that assemble and form a solid structure following injection. The sutures maintain the tendon in a desired position and are suitable to maintain the implant 404, 504, 1204 Repair material 1224 is then injected into the delivery device with the implant 404, 504, 1204 prior to or during injection of the implant 404, 504, 1204 into the repair site 1292 utilizing the same delivery device 1216. In another embodiment, the implant 404, 504, 1204 is pretreated with repair material to form a mixture prior to insertion into the repair site 1292.

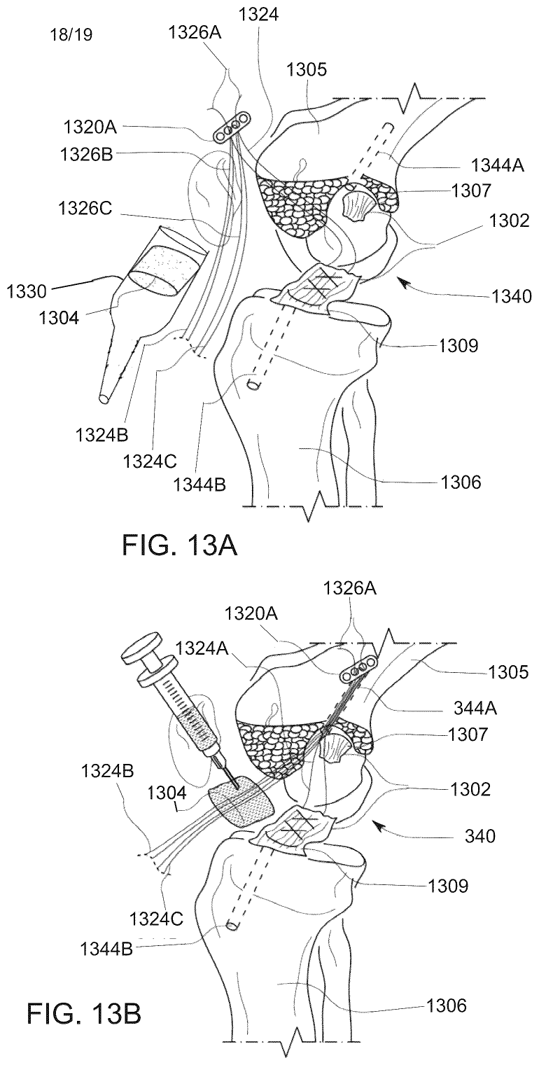

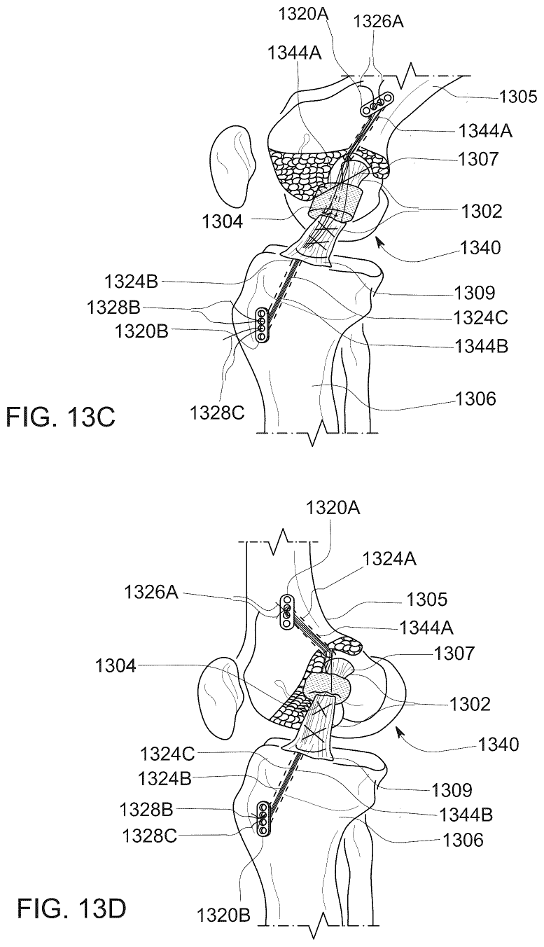

Another example of a ruptured ACL is depicted in FIGS. 13A-13D. The ACL 1302 is one of four strong ligaments that connects the bones of the knee joint. The function of the ACL is to provide stability to the knee and minimize stress across the knee joint. It restrains excessive forward movement of the lower leg bone, the tibia 1306, in relation to the thigh bone, the femur 204, and limits the rotational movements of the knee.

The ACL 1302 is ruptured such that it no longer forms a connection between the femur bone 1305 and the tibia bone 1306. The resulting ends of the ruptured ACL 1302 may be of any length. The ends may be of a similar length, or one end may be longer in length than the other. The end on the femur 1305 includes the femoral ACL stump 1307. The end on the tibia 1306 includes a tibial stump 1309. In some instances, it is believed that a repair is desirable when the tibial stump length SL is less than about 75% of the effective ligament length LL but greater than 5% of a total length LL of the ACL. The total length of the ACL is considered to be the length of ligament from femoral footprint to the tibial footprint along a linear axis.

The knee joint includes tibial spines on the tibia 1306 and the intercondylar notch of the femur 1305. In some instances, the methods as described herein may include performing a notchplasty of the intercondylar notch of the femur to provide space for larger ligament to form after surgical repair using a scaffold. Such a notchplasty improves the size of the healing ligament, specifically resulting in a larger cross-sectional area of the ligament. As the mechanical strength of a ligament, and subsequently its ability to maintain the distance between the femur and tibia, is directly correlated with its cross sectional area, enlarging the notch with a notchplasty can help make a stronger repaired ACL and has been found by the inventors to be beneficial in ACL repair using a scaffold as described in the present disclosure.

Aspects of the invention provide methods of repairing the ruptured ligament 1302 involving drilling one or more holes 1344 at or near the repair site 1340 of the ruptured ligament 1302. A bone at or near a repair site is one that is within close proximity to the repair site and can be utilized using the methods and devices of the invention. For example, the bone at or near a repair site of a torn anterior cruciate ligament is the femur 1305 bone and/or the tibia 1306 bone. The hole 1344 can be drilled into a bone using a device such as a Kirschner wire (for example a small Kirschner wire) and drill, or microfracture pics or awls. One or more holes may be drilled into a bone surrounding the repair site 1340 to promote bleeding into the repair site 1340. The repair can be supplemented by drilling holes into the surrounding bone to cause bleeding. Encouraging bleeding into the repair site may promote the formation of blood clots and enhance the healing process of the injury.

In FIG. 13A, holes 1344A, 1344B are drilled into the femur 1305 and the tibia 1306, respectively, at the repair site 1340. The hole 1344A may be additionally referred to hereinafter as the femoral tunnel 1344A and the hole 1344B may be additionally referred to hereinafter as the tibial tunnel 1344B A first suture 1324A is placed through the tibial stump 1309 using a whip-stitch. The first suture 1324A is attached via the first end 1326A to a first fixation device 1320A. A second suture 1324B and a third suture 1324C are coupled to the fixation device 1320A at respective first ends 1326B, 1326C. The fixation device 1320A is subsequently passed through the femoral tunnel 1344A and coupled to the femur 1305.

In FIG. 13B, the implant 404, 504, 1304 is loaded onto the second and third sutures 1324B, 1324C. The implant 404, 504, 1304 is then injected with repair material as described. The implant 404, 504, 1304 and the second and third sutures 1324 may be inserted into the repair site 1340 via the delivery device 1330. In FIG. 13C, the free ends 1328B, 1328C of the second and third sutures 1324B, 1324C are passed through the tibial tunnel 1344B and are coupled to a second fixation device 1320B coupled to the tibia 1306. The implant 404, 504, 1304 is then positioned between the two ends of the torn ACL 1302. In FIG. 13D, the knee is extended and the sutures 1324A, 1324B, 1324C, and the fixation devices 1320A, 1320B are secured. As described here and in the embodiments below, the first and second fixation devices may be anchors. In another embodiment, the first and second fixation devices may be screws, barbs, or extracortical bone fixation devices.

In another embodiment, the implant 404, 504, 1304 may be indirectly coupled to the first and second fixation devices 1320A, 1320B and held in position in the repair site 1340 by additional sutures 1324. In addition, in another embodiment, any of the first, second, or additional sutures 1324A, 1324B, . . . 1324n may be attached to one or both ends of a ruptured ligament 1302 by their first ends 1326A, 1326B, . . . 1326n and/or their second ends 1328A, 1328B, . . . 1328n. Furthermore, in another embodiment, additional fixation devices 1320 and sutures 1324 may be directly or indirectly attached to either the tibia bone 1306 or the femur bone 1305 to secure the implant 404, 504, 1304 in position. In alternative embodiments, the implant 404, 504, 1304 may be attached to the femur bone 1305 directly or indirectly.

In yet another embodiment, a tibial tunnel is drilled in a tibia. A femoral tunnel is drilled in a femur so that the femoral tunnel and tibia tunnels are aligned. An extracortical bone fixation device is placed on a femoral cortex. A first and second set of sutures are attached to the bone fixation device and pass through the femoral tunnel. The first set of sutures are secured onto the ACL having a rupture or tear. A collagen implant is threaded along or on the second set of sutures. A volume of blood is injected into the collagen implant so that the blood is positioned at or adjacent the rupture or tear of the ACL and a rupture or tear of the meniscus. The first and second set of sutures, attached to a second bone fixation device, are passed through the tibial tunnel. The second bone fixation device is secured in place on the tibial cortex. Growth of ACL tissue and meniscus at or near a rupture or tear of the ACL and the meniscus is observed. This includes obtaining and analyzing an MRI image of the ACL tissue and meniscus at the tissue site and analyzing the MRI image to identify growth of the ACL tissue and meniscus.

In yet another embodiment, a first set of sutures and a second set of sutures are coupled to a bone fixation device. A first portion of the first set of sutures is secured to a ruptured or torn anterior cruciate ligament. The bone fixation device is positioned on a surface of the femur so that the first and second sets of sutures extend through a femoral tunnel. A collagen implant is positioned along the second set of sutures. A volume of blood is injected into the collagen implant so that the blood is proximate the ACL and a lateral or medial meniscus. The second set of sutures is secured to a surface of the tibia so that the second set of sutures extending through a tibial tunnel with a second bone fixation device. Growth of an ACL tissue and meniscus at or near a rupture or tear of the ACL and the meniscus is observed. This includes obtaining and analyzing an MRI image of the ACL tissue and meniscus at the tissue site and analyzing the MRI image to identify growth of the ACL tissue and meniscus.

In yet another embodiment, a first portion of the first set of sutures are secured to a ruptured or torn anterior cruciate ligament. A second portion of the first set of sutures are secured relative to the femur such that the first set of sutures extends from the ruptured or torn anterior cruciate ligament and through into the femoral tunnel to the bone fixation device. A first portion of the second set of sutures are secured relative to the tibia so that a suture tail of the second set of sutures extends through a tibial tunnel into the region near the anterior cruciate ligament. A second portion of the second set of sutures are secured relative to a femur at the bone fixation device so that the second set of sutures extends into a femoral tibia tunnel of the tibia alongside or adjacent the anterior cruciate ligament. A volume of blood is injected into a collagen implant. The collagen implant, with the volume of blood absorbed therein, is threaded along the second set of suture to a location adjacent the rupture or torn portion of the anterior cruciate ligament. Blood from the collagen implant is permitted to emanate from the collagen implant into the region near the anterior cruciate ligament and a tear or rupture in either or both of a lateral meniscus and a medial meniscus. Healing of the rupture or tear of ACL and the lateral meniscus or medial meniscus is observed. Growth of ACL tissue and meniscus tissue at or near a rupture or tear of the ACL and the meniscus is further observed. This includes obtaining and analyzing an MRI image of the ACL tissue and meniscus at the tissue site and analyzing the MRI image to identify growth of the ACL tissue and meniscus.

Further described herein are methods for repairing a meniscus. Such methods may include forming a portal in a knee joint to access a lateral or medial meniscus, wherein the lateral or medial meniscus has a rupture or tear. In embodiments, the methods include inserting a delivery device into the portal so that a distal end of the delivery device is positioned close to the lateral or medial meniscus.

Methods for repairing a meniscus may further include causing a collagen implant according to various embodiments herein to exit the distal end of the delivery device in order to position it on or near the rupture or tear of the lateral or medial meniscus. Subsequently, the collagen implant may be secured in place at the rupture or tear of the lateral or medial meniscus.

In one or more embodiments, the methods of repairing the meniscus may include combining the collagen implant with blood prior to inserting the delivery device into the portal. In some embodiments, the blood comprises autologous blood.

In various embodiments the methods include securing a first set of sutures onto the meniscus. Embodiments may further include threading the collagen implant along or on a second set of sutures. The collagen implant may be positioned between a ruptured portion of the meniscus via the first and second sets of sutures.

### EXAMPLES

### Example 1—Preparation of a Collagen Implant

Surgical rotator cuff repair is conducted when non-surgical efforts do not yield the desired results. In principle during the surgery, an implant is placed in, on or over the injury site to bridge the tear in the rotator cuff. The approach is either open, mini-open or arthroscopic, each with its own variation in surgical technique. Known challenges with rotator cuff repair relate to implant deployment and placement, due to confined space, especially in mini-op and arthroscopic repair.

The collagen implants investigated here included injectable powders, injectable pieces, and semi-solid or solid implants of various shapes. Collagen implants were prepared utilizing the manufacturing process described in U.S. Pat. No. 11,826,489, which is incorporated by reference herein in its entirety.

Extraction process: To be usable for surgery in human or veterinary medicine, all elements that the receiving host could recognize as foreign were removed. This was achieved via a series of steps, including:
1. Salt extraction to remove unwanted DNA.
2. Using high concentrations of sodium chloride, approximately 5 M in water or buffer system (physiological pH, for example TRIS or others)
3. Detergent extraction to disrupt cell membranes (lysis) and extract proteins. The detergents used included at least one of Sodium Dodecyl Sulphate (SDS), Triton X-100, Triton X-104, Tween 20, Tween 8, and/or CHAPS.
4. Enzymatic digestion: DNAse and RNAse were use to cleave and remove DNA and RNA from the mammalian tissue at physiological pH. The tissue was contacted with pepsin at low pH.
5. Acid solubilization: Hydrochloric Acid was used at high concentrations and low pH.
6. Intermittent water and/or buffer rinses (preferably at physiological pH and salt concentrations) were performed prior to and/or after each step.

Manufacturing Process Molding—Lyophilization: The final slurry formed by the extraction process may then be processed through lyophilization into a dried semi-solid or solid collagen component. The final configuration of the implant was created generally by the following steps:
1. Final composition containing purified collagen, and other useful components for treating and/or repairing a tissue, and providing desirable structural and dissolution characteristics
2. Gelation
3. Molding
4. Freeze drying
5. Cutting, chopping, grinding, or milling
6. Absorption

Final composition: Once the extraction process was completed, the resulting purified collagen was adjusted to the desired concentration in a range of 10 g/L to 80 g/L, 400 mg/g to 10,000 mg/g, or 40 w/w % to 70 w/w %, depending on the application (e.g., torn or ruptured meniscus, ACL, rotator cuff, or combinations thereof). In addition to the purified collagen, one of the following materials was added to the purified collagen:
1. A salt such as calcium, magnesium, other salts, or combinations thereof, which are able to facilitate healing of the ruptured or torn tissue.
2. One or more factors to improve the outcome/healing process post-surgery, for example: growth factors, proteins, e.g., PDGF, VEGF, TGF-ß, peptide chains, e.g., CU-GHK, RGD-peptides, proteinase and/or collagenase inhibitors, other molecules know to improve wound healing, e.g., Zn-ions, retinol, vitamins, steroids.
3. Variations of the other components described within this disclosure.

A non-limiting example of a collagen implant composition was prepared having the components shown in Table 1. Those skilled in the art will understand that this example is non-limiting. Other combinations of the described components and/or variations in the composition mixture and amounts in accordance with the entirety of this disclosure are also suitable for implant compositions according to the invention.

TABLE 1
Collagen Implant Composition for forming the Collagen Implants

| | Composition | |
|---|---|---|
| Component | (dry in w/w %) | Range |
| Collagen | 65% | 40 to 70% |
| HEPES | 21% | As needed |
| Calcium | 3% | 2-5% |
| Sodium | 3% | 2-5% |
| Chloride | 7% | 5-11% |
| GAG | <1% | 0-2% |

Gelation: In some instances, a controlled self-assembly of the purified collagen molecules in the implant composition was implemented. The self-assembly process was thermally driven and occurred faster at higher temperatures. The progress of self-assembly was monitored by measuring the viscosity of the material, increasing over time. In this example, the viscosity was equivalent to a soft-boiled egg yolk (e.g., about 0.0181 Pa/s to about 0.0304 Pa/s). To achieve the desired amount of self-assembly, the temperature of the implant composition may be increased from room temperature to about 30° C. to about 37° C. for a period of one hour and up to 8 hours. In this example, the self-assembly step was conducted at 34° C. for a duration of 180 minutes.

Self-assembly and gelation influences the resistance of the implant to resorption in the host and by extension how long the device persists in the body. There is no direct correlation of this, other than even smaller amounts of gelation result in the implant being more stable in the body. Controlled self-assembly and gelation were performed using the following processes:

Gelation Method A: Pieces with Defined Dimensions

In this example, the implant composition was poured into a metal tray to form a sheet having a thickness of 1 cm to 4 cm. The tray containing the implant composition was then placed in a chamber at 35° C. for 120 min to achieve the desired self-assembly. Afterwards, the tray was transferred into a fridge at 4° C. or lower to cool the material down and slow down the self-assembly process to an essential standstill. The material was then placed in a lyophilizer and freeze dried until achieving a residual moisture level of 3 w/w % to 5 w/w %.

Gelation Method B: Pieces with Pre-Determined Defined Dimensions

In this example, the implant composition was poured into a metal tray having a grid received therein to separate the tray into areas of identical size. The grid was formed of a thin metal sheet, similar to an ice cube tray. The tray was configured to form cuboid portions of the implant composition, each having a thickness of 1 cm to 4 cm. The tray containing the implant composition was then placed in a chamber at 35° C. for 120 min to achieve the desired self-assembly. Afterwards, the tray was transferred into a refrigerator at 4° C. or lower to cool the material down and slow down the self-assembly process to an essential standstill. The material was then placed in a lyophilizer and freeze dried until achieving a residual moisture level of 3 w/w % to 5 w/w %.
Gelation Example B.1: In one configuration, the grid was part of the tray, such that the individual segments were filled with a defined volume of the implant composition to achieve a final amount of material of each cuboid to be 2.0 g.
Gelation Example B.2: In another configuration, the grid was a separate component and inserted into the tray after the material was poured into it. That way the material was separated into equal weighted aliquots by the metal sheets making up the grid.

Thermal re-assembly (Gelation) may be further employed to achieve self-assembly of the solubilized collagen to control the ability and speed of the implant to form a suspension with blood. No gelation results in fast blood absorption. Gelation for 180 min at 34° C. resulted in blood absorption over approximately 4 min. The resorption profile of the implant during the healing process also may be impacted by gelation. Gelation for 150 min at 35° C. results in an absorption time of approximately 35 to 42 days.

The trays were then placed in a chamber at 32° C. for 240 mins to achieve the desired amount of self-assembly. Afterwards, the tray was transferred into a refrigerator at 4° C. or lower to cool the material down and slow down the self-assembly process to an essential standstill. The material was then placed in a lyophilizer and freeze dried to achieve a residual moisture level of less than 2 w/w %.

There are many possible variations of the lyophilization tray that can be implemented. For example, the tray and/or grid can form a three-dimensional shape based on any geometry such as a square, rectangle, circle, oval, hexagon, rhomboid or may even be irregularly shaped. The floor of the tray may be an integral part of the tray or may be separatable from the outer frame and/or the inner grid if it exists. The inner grid can be made of a single piece with a fixed, defined size of the compartments therein or made up to form individual bars that allow for multiple varied sizes of compartments within the tray. The inner grid may be configured such that it does not extend all the way through the material from the bottom of the tray, but only partially, for example 30% to 50% of the depth. In this case, the sheets can be broken into pieces of a desired size after lyophilization, similar to chocolate bars.

Final shaping of the implant: Several variations of a process for shaping an injectable collagen implant were investigated. The size of the semi-solid or solid component, particles, pieces etc. was varied in consideration of injecting the implant from a variety of syringes and cannula sizes after absorption with the patient's own blood.

### Removal Process

The configuration of the lyophilization tray drives the removal process of the dried material from within it. In one embodiment, the tray was turned upside down and either the sheet or the portions of a defined size were released.

In another method, the dried implants each having a defined size were pushed or pulled out of the mold either manually or by a tool. Alternatively the tray or the individual wells were coated with a removable lining that released the sheet or the defined portions when removed.

The trays, if made from a flexible material (e.g., silicone), can be pulled onto a drum with the opening of the tray or wells facing outwards to release the sheet or the individual portions by expanding the material of the tray/walls of each well so that gravitational forces are sufficient to remove them.

### Portioning of the Material to the Desired Amount

This optional step was advantageous after forming lyophilized sheets that could be subsequently cut or divided to form a plurality of pieces or particles having a desired mean size. In the case of a tray having individual wells that lyophilize the material in predetermined amounts, this step may be omitted because the nature of the tray and its wells ensures the correct amount for each piece. If the material is lyophilized as a single sheet, then the material may first be separated into smaller pieces, for example, by cutting with a blade/scissors, a bandsaw-like device, a die cutter, or similar laboratory equipment. The die cutter has the advantage that it would directly create pieces of the final desired size (portion).

In case of the tray having compartments whose walls do not fully partition the material, but form areas of thinner material thickness, thereby forming pre-determined break lines, similar to a bar of chocolate, the material is broken apart. The breaking occurs at the areas of thinner material and can be facilitated by breaking over an edge/blade or a corner, or cutting with a blade or similar device. In one example, the breaking occurs first in one direction than in another, following the areas of thinner material. The final pieces may be of a defined range or size, geometry and weight.

In an alternate tray design, there are compartments, but parts of the walls extend all the way through the material creating areas along the walls of the compartment where there is no material and areas where the material is less in thickness than in the bulk of the material. This would look similar to a bar of chocolate with perforations between the pieces. In subsequent steps, the material may be broken into pieces of defined weight, size and geometry. In the example of a tray with separated compartments, this step can be omitted if the compartments are of a size that matches the final desired size, for example, a final desired particle size of greater than 1 mm (in any dimension) to 100 mm (in any dimension). If the step is performed, it may be performed with partitioned materials to control the final amount. The material may be cut, sliced, grated, shredded, ground, or milled, or any combination thereof. If a defined particle size or particle size range is desired, size exclusion separation techniques may be employed. If this is not required, the material may be used without size exclusion techniques.

It was determined that the portioned implants could be fed into a cylinder having one or more rotating blades therein. Depending on the speed of the blades and the rate of feeding the material, the resulting particles may vary in size. Boths parameters can be used to control the particle size. The material may alternatively be fed into a container with rotating blades at the bottom. Cutting would be controlled similar to food processing by time, pulse, speed, and/or blade design. The cutting and size exclusion separation may be combined by using a rotating blade that is fundamentally round and has integrated blades. Such blades are configured to continually shave off material from the larger piece of material with the material falling down through the blade similar to food grinders or shredders. The cut size may be controlled by the rise of the cutting blade above the level of the of the plane of the blade's rotation and can vary widely (up to 10 mm or more), and by the shape of the blade (straight, concave, convex, angled, v or u-shaped) relative to the plane of the blade's rotation.

### Cutting Example A (Continued from Gelation)

Cutting: The resulting collagen sheet was removed from the tray and then cut into pieces of a certain size to yield the required mass for the device, for example 2.0 g. The exact mass is determined by the desired application and can be anywhere from a few micrograms up to several hundreds of grams. The smaller, uniformly weighted pieces, representing the final weight of the device were then cut with a blade or a band saw into smaller pieces of approximately 3 mm×3 mm×3 mm. Notably, the size of these pieces may be varied depending on the requirements of the application. All of the pieces were then transferred into a glass vial, which was sealed and then sterilized.

### Cutting Example B (Continued from Gelation)

No further sizing: In both examples the dry material is then removed from its compartments, yielding pieces of a desired and pre-determined size for the intended application, for example 5 mm×5 mm×5 mm. A defined number of these pieces, for example 15 to yield a final weight 1.8 g are then filled into a plastic vial, sealed and sterilized.

Grating: Cut sheet into strips, then feed strips into a hopper with rotating bladed at the bottom, cutting the strips into slices (see FIG. 4 for examples). Thickness of slices is defined by the speed of the blade and the advancing speed of the strip into the path of the blade (similar to extrusion processes). This can yield very thin slices (shavings) or thicker slices. Generally the thickness of the slices is less than their circumference.

Blending/shredding: Implant sheets formed by lyophilization may be cut into smaller pieces (e.g., undefined size, defined size) and then placed into a container with rotating blades in the bottom, similar to a food processor. To control the resulting particle size, the following parameters may be varied: blade design, length, width, geometry (straight, bent, wavy), cross-section, blade speed (e.g., from 10 revs/s to 50,000 revs/s), blending duration (e.g., continuous from 0.5 s to 15 s or more), pulsed with similar durations as above. The container may be cooled to prevent any heat damage to the material form the exposure to the process.

Grinding/Milling: The implants may be pre-cut into smaller pieces of an undefined size and then fed in batches into a mill to grind the material down to form a powder. Any type of mill is suitable (e.g., a ball mill or a hammer mill). Using a ball mill, the following parameters may be controlled to achieve the desired result: number of balls in chamber, size of balls in the chamber (notably, the balls do not need to be of a single size), materials of balls in the chamber (note the balls do not all need to be of the same material), ratio of ball size/number to chamber size, speed of rotation of chamber, and/or the chamber might need to be cooled to approximately 4° C. to avoid thermal material damage. Using a hammer mill, the following parameters may need to be controlled to achieve the desired result: hammer speed, amount of material filled into the chamber, distance of hammer from the frame, and/or the chamber may need to be cooled (e.g., to 4° C.) to prevent heat damage to the material. In order to keep the material cool and to increase its brittleness, dry ice or liquid nitrogen was added to the chamber. Both were removed subsequently via simple evaporation.

Size Exclusion Separation (SES): After the milling, cutting, grating, grinding, etc. process, the resulting particles were separated into a desired particle size, depending on the application. In an extreme case, the application may have no requirements for a desired particle size and no size exclusion separation technique may be needed. Four (4) SES methods were evaluated: to achieve a mix of particles with a defined minimum size (low cut off), to achieve a mix of particles with a defined maximum size (high cut off), to achieve a mix of particles with a defined minimum and maximum size (range), and/or to combine more than one size range of particles, if that is required (e.g., a combination of particles with a range of 50 μm to 400 μm and 800 μm to 900 μm, a combination of particles no bigger than 250 μm and no smaller than 500 μm), or combinations thereof. Various forms of sieving were investigated:

Dry sieving: Dry sieving is commonly used for particle size analysis and separation. It involved passing the material through the sieve with openings of varied sizes. The finer particles passed through the sieve, while larger particles were retained. This method was highly efficient and widely employed due to its simplicity and reliability.

Vibratory sieving: Vibratory sieves utilized vibrations to enhance the sieving process. The sieve was subjected to oscillatory movements, causing the material to move rapidly, facilitating separation. Vibratory sieving was highly effective in achieving precise and efficient separation, particularly for fine powders and granules. Its ability to minimize blinding and improve throughput makes it a popular choice in industries such as pharmaceuticals and chemical processing.

Centrifugal Sieving: Centrifugal sieving involved the use of centrifugal force to enhance the separation process. The sieve was rotated at high speeds, creating a centrifugal acceleration that aids in the separation of particles based on size and density. This technique was advantageous for achieving high throughput and accurate separation when fine particles and powdered materials were needed.

Air Jet Sieving: Air jet sieving employed a stream of air to fluidize the material sieved, allowing the finer particles to pass through the sieve. Choosing stainless steel sieve screens for particle analysis helped the air jet effectively disperse and separate the particles based on their size, resulting in precise classification. This technique was commonly used in industries where high accuracy and repeatability are crucial, such as the pharmaceutical and food industries.

Tapping Sieving: Tapping sieving utilized mechanical tapping or striking action to assist in the sieving process. Operators subjected the sieve to tapping or knocking, which assisted in breaking down agglomerates and improving separation efficiency. Industries, such as construction, mining, and pharmaceuticals, widely utilize this technique for particle size analysis. This method was well suited to achieve a defined particle size distribution by arranging a series of sieves with different cut-offs in sequence (stacked on top of each other).

Rotary Sieving: Rotary sieving involved the use of a rotating sieve drum to separate materials based on size. Operators fed the material into the drum, and as it rotated, the sieve openings allowed smaller particles to pass through while retaining larger particles. Rotary sieves are versatile and widely employed in various industries for efficient particle separation.

Multilayer Sieving: Multilayer sieving involved using multiple layers of sieves stacked on top of each other to achieve more precise separation. Each sieve layer had different openings, allowing for finer classification of materials. Industries, such as pharmaceuticals and fine chemicals, employ multilayer sieving when high precision and accurate separation are of utmost importance.

Combination of milling and SES: The following process created smaller particles from the lyophilized sheet and by combining milling and sieving methods to achieve the desired particle size in a single processing step. A hammer mill was used having a limited hammer speed, while the system was cooled to prevent heat damage during milling. An implant sheet was fed into a blender to create smaller pieces, then the smaller pieces were transferred into a grinder to get them to a desired particle size, for example, 500 μm to 2,000 μm. The sheet was fed into a blender to create smaller pieces, then the smaller pieces were transferred into a mill to grind them to a desired particle size, for example 50 μm to 120 μm, or 10 to 50 μm.

### Further Variations to the Implant Compositions to Achieve Desired Properties

Viscosity: The final properties of the implant composition and/or implant (e.g., semi-solid, solid, injectable) may be determined by the final viscosity. The collagen implant may be divided into particles, flakes, chunks, etc. and suspended or gelled in blood (itself a suspension) having a desired viscosity. The manufacturing process controls the solid element of the final suspension being applied, mostly in terms of size and shape. The amount of blood used is defined by the requirements of the surgical procedure, mostly in terms of volume. Other exemplary considerations include blood coagulation, heparinized blood (slowest), heparinized blood with calcium ions added (slow), whole blood (faster), whole blood with Calcium added (fastest). As a result viscosity can be varied broadly by changing one or more of these parameters.

Porosity: The porosity of the collagen particles is related to the amount of fluid (e.g., blood) that they can absorb. The bigger the particles the more important porosity of the particles becomes for application. Blood absorption into collagen is suitable to turn the material into a suspension, making the solids malleable (gel-like) and suitable to push the suspension through a syringe. Such suspensions may allow larger particles to go through a narrower opening. Implant materials evaluated herein had a porosity of about 80% to about 99%. Generally, the smaller the particles, the less important the porosity becomes. For particles smaller than 1 mm, a porosity of 10% or less may be possible.

### Applicator

The applicator employed for ejection of the implant, had a tubular structure of sufficient length to reach the desired anatomical site, and sufficient diameter to apply the blood saturated implant with an ample volume and viscosity to fill the desired space. The means of advancing/ejecting the collagen implant was a longitudinal piece that was inserted into the tubular structure from one end and by advancing it along the length of the tubular structure displacing the collagen implant out of the other end of the tubular structure. Notably, the tubular structure can be of any cross section and the inner and outer diameter can be consistent or variable along its length (e.g., the diameter of the tubular structure can narrow towards the exits to allow for better control of the placement of the collagen implant). The longitudinal piece may be solid and of a constant cross-section or it could have a variable cross-section. The material of construction may be suitable to allow for adjustment of its diameter to the inner diameter and geometry of the tubular structure. In this example, the applicator was a syringe. During the surgical procedure, the collagen implant was placed in the barrel of the syringe and then a defined amount of the patient's own blood was added to the barrel of the syringe. The barrel had a marking to indicate the volume of blood required (range, min, maximum amount or combinations thereof). Notably, the applicator can include, for example as part of the piston, a means (e.g., a propeller, baffles, internal mixer, etc.) to mix the collagen implant and the autologous blood in the barrel of the syringe.

### Example 2—Surgical Repair of a Tissue Using a Collagen Implant

In this example, the collagen implant was comprised of particles have a mean size of about 500 μm to about 2,000 μm with a total mass of 1.8 g. The applicator was a syringe with a volume of 20 ml, the barrel of the syringe and the piston being separated. The collagen implant was filled into the barrel of the syringe, which in turn was sealed at the top and the bottom.

The filled barrel of the syringe and its matching piston were placed in a tray, designed to physically separate the two components and keep them stable in place by means of two deep drawn cavities in a plastic blister. The blister was sealed with an air- and moisture barrier, that is, a standard foil material used in medical packaging. The blister was placed in a carton which was sufficiently strong to protect the product during transit from any damage or loss of seal integrity.

During surgery, the surgeon removed the seal from the back end of the syringe and filled between 4 ml and 7 ml of the patient's own blood into the barrel of the syringe. Subsequently, the piston was inserted into the barrel. Shaking the applicator achieved the desired mixing of the blood and the collagen implant to prepare the implant for use.

To place the implant, the seal at the front of the syringe was removed. If required, a cannula was attached to the front of the syringe to facilitate the delivery of the, now blood saturated collagen implant, into difficult to reach anatomical spaces. The implant was placed by depressing the plunger and ejecting the implant (through the cannula) into the desired anatomical location. The size of the cannula was chosen to facilitate ejection of the implant. In this example, the collagen implant was in the form of compressible particles absorbed with the patient's blood and suitable for ejection through cannula having a diameter of greater than about 2,000 μm. The collagen implant also could have been in the form of a single semi-solid or solid piece that is compressible and deformable, configured for ejection through a suitable sized cannula.

### Example 3—Surgical Repair of a Tissue Using a Collagen Implant

In this example, the collagen implant was comprised of particles with a mean size of less than 2,500 μm and a total mass of 1.5 g. The collagen implant was filled into a glass vial with a hermetic seal and a narrow neck opening to facilitate the process of removing the implant form the vial and into the applicator without a funnel.

The applicator was an open bore syringe with a volume of 25 ml. The open bore at the lower end was sealed by a screwcap.

Both components were placed in in a folded paper tray with two compartments to keep the vial and the applicator separate. The paper tray was equipped with a lid (similar to a regular gift box or folding box) and protected the components during transit.

During surgical use, the applicator was prepared by removing the piston from the syringe and putting it aside. Then the vial was opened and the collagen implant was directly filled into the barrel of the syringe. Subsequently, 8 ml of autologous blood were filled into the barrel of the syringe and the piston was inserted back into the barrel. Shaking the applicator achieved the desired mixing of the blood and the collagen implant to prepare the implant for use.

To place the implant, the screwcap at the front of the applicator was removed, the applicator was then ready for use. The surgeon inserted the applicator and guided it to the desired anatomical space and then injected the implant by depressing the plunger. The size of the cannula was chosen to facilitate ejection of the implant. In this example, the collagen implant was in the form of compressible particles absorbed with the patient's blood and suitable for ejection through cannula having a diameter of greater than about 2,500 μm. The collagen implant also could have been in the form of a single semi-solid or solid piece that is compressible and deformable, configured for ejection through a suitable sized cannula.

In the present disclosure, a subject includes, but is not limited to, any mammal, such as human, non-human primate, mouse, rat, dog, cat, horse or cow. In certain embodiments, a subject is a human. The present disclosure may also include kits for repair of ruptured or torn meniscus and cartilage. A kit may include an implant of the invention having at least one delivery device that carries that collagen implant and instructions for use. The implant may further include one or more sutures that attach a fixation device to the implant. A kit may further include a container that contains a repair material as described herein.

The foregoing written description is considered to be sufficient to enable one skilled in the art to practice the invention. The present disclosure is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention. Those skilled in the art will recognize or be able to ascertain using no more than routine experimentation, many equivalents to the specific embodiments of the invention described herein. Such equivalents are intended to be encompassed by the following claims.

1-29. (canceled)

30. A method for repairing a rotator cuff tendon, comprising:
attaching a first fixation device to a humerus;
attaching a second fixation device to the rotator cuff tendon;
connecting a flexible construct to the two fixation devices;
injecting a collagen implant on or near the rotator cuff such that the collagen implant rests between torn ends of the rotator cuff tendon.

31. The method of claim 30, further comprising attaching the first fixation device only indirectly to the collagen implant.

32. The method of claim 30, further comprising positioning the collagen implant between a ruptured portion of the rotator cuff via the flexible constructs.

33. The method of claim 30, further comprising combining the collagen implant with blood prior to injecting the collagen implant.

34. The method of claim 33, wherein the blood comprises autologous blood.

35. The method of claim 30, further comprising adding the collagen implant to the delivery device, and adding blood to the delivery device prior to inserting a delivery device through a portal.

36. The method of claim 30, wherein the collagen implant comprises heterotrimeric collagen, tropocollagen, atelocollagen, fibrillar collagen, or combinations thereof.

37. The method of claim 30, wherein the collagen implant comprises Type I collagen.

38. The method of claim 37, wherein Type I collagen comprises heterotrimeric collagen having two α1(I) chains, and one α2(I) chain, the α1(I) chains being about 100 nm to about 500 nm long.

39. The method of claim 30, wherein the collagen is derived from rat, pig, cow, or human tissue.

40. The method of claim 30, wherein the collagen implant does not comprise cross-linked collagen.

41. The method of claim 30, wherein the collagen implant comprises a buffer comprising 4-(2-hydroxyethyl)-1-piperazineethanesulfonic acid (HEPES), tris(hydroxymethyl) aminomethane) (TRIS), (3-(N-morpholino)propanesulfonic acid) (MOPS), 3-[N-Tris(hydroxymethyl)methylamino]-2-hydroxypropanesulfonic acid (TAPSO), cacodylate SSC, succinic acid, glycine, sodium phosphate, sodium hydroxide, sodium chloride, magnesium chloride, acetic acid, thrombin buffer, L-ascorbic acid phosphate magnesium salt n-hydrate, MMP-1, MMP-2, MMP-3, MMP-9, RNAase/DNAase, elastase, papain, citrate, sodium citrate, phosphate, saline, or combinations thereof.

42. The method of claim 30, wherein the collagen implant further comprises a zwitterionic buffer.

43. The method of claim 42, wherein the zwitterionic buffer comprises 4-(2-hydroxyethyl)-1-piperazineethanesulfonic acid (HEPES), betaine, tris(hydroxymethyl)aminomethane) (TRIS), (3-(N-morpholino)propanesulfonic acid) (MOPS), (3-(Cyclohexylamino)-1-propanesulfonic acid) (CAPS), (2-(N-morpholino) ethanesulfonic acid) (MES), 2,2′-[(2-amino-2-oxoethyl)azanediyl]diacetic acid (ADS), 2,2′-(Piperazine-1,4-diyl)di(ethane-1-sulfonic acid) (PIPES), 2-[(2-Amino-2-oxoethyl)amino]ethane-1-sulfonic acid (ACES), 2-Hydroxy-3-(morpholin-4-yl)propane-1-sulfonic acid (MOPSO), cholamine chloride hydrochloride, N,N-Bis(2-hydroxyethyl)taurine (BES), 2-{[1,3-Dihydroxy-2-(hydroxymethyl)propan-2-yl]amino} ethane-1-sulfonic acid (TES), 3-(N,N-Bis(2-Hydroxyethyl)amino) (DIPSO), 3-[[1,3-dihydroxy-2-(hydroxymethyl)propan-2-yl]amino]-2-hydroxypropane-1-sulfonic acid (TAPSO), acetamido-glycine, Piperazine-1,4-bis(2-hydroxy-3-propanesulfonic acid),dihydrate (POPSO), N-(2-Hydroxyethyl)piperazine-N′-(3-propanesulfonic acid) (HEPPSO), 3-[4-(2-Hydroxyethyl) piperazin-1-yl]propane-1-sulfonic acid (HEPPS), N-[1,3-Dihydroxy-2-(hydroxymethyl)propan-2-yl]glycine (Tricine), glycinamide, glycylglycine, bicine, 3-{[1,3-Dihydroxy-2-(hydroxymethyl) propan-2-yl]amino}propane-1-sulfonic acid (TAPS), or combinations thereof.

44. The method of claim 30, wherein the collagen implant further comprises one or more salts comprising calcium containing salts, magnesium containing salts, sodium containing salts, calcium chloride, magnesium chloride, sodium chloride, or combinations thereof.

45. The method of claim 30, wherein the collagen implant comprises an electrolyte comprising chloride, nitrate, sulfate, silicate, phosphate, aluminate, sodium tetraborate, or combinations thereof.

46. The method of claim 30, wherein the collagen implant comprises one or more glycosaminoglycans comprising heparan sulfate, chondroitin sulfate, dermatan sulfate, hyaluronic acid, keratan sulfate, or combinations thereof.

47. The method of claim 30, wherein the collagen implant comprises a plurality of particles comprising pieces, granules, extrudates, powders, pellets, multi-particulates, minitablets, microcapsules, cuboids, hexoids, rhomboids, spheres, microspheres, cylinders, ovals, fibers, or combinations thereof.

48. The method of claim 30, wherein the mean size of each of the plurality of particles is less than about 100 mm.

49. The method of claim 30, wherein the plurality of particles each comprises a porosity of about 50% to about 99%.
